SEQUENCE LISTING (4 sequences)

SEQ ID NO: 1 - DNA, 431 bp, Artificial Sequence
  SYNTHESIZED
gaatacatcaggcaggcaagggcatcaagatgaagtcacagacccaggtcttcgtatttctactgctctgtgtgtctggtgctcatgggagtattgtgatgacccagactcccaaattcctgcttgtctcagcaggagacaggattaccataacctgcaaggccagtcagagtgtgaataatgatgtagcttggtaccaacagaagccagggcagtctcctaaactgctgatatactatgcatccaatcgctacactggagtccctgatcgcttcactggcagtggatatgggacggatttcactttcaccatcaccactgtgaaggctgaagacccggcagtttatttctgtcagcaggattatagctctccgctcacgttcggtgctgggaccaagctggaaatcaaacgtaagtcgactgcacaactg

SEQ ID NO: 2 - protein, 143 aa, Artificial Sequence
  SYNTHESIZED
IHQAGKGIKMKSQTQVFVFLLLCVSGAHGSIVMTQTPKFLLVSAGDRITITCKASQSVNNDVAWYQQKPGQSPKLLIYYASNRYTGVPDRFTGSGYGTDFTFTITTVKAEDPAVYFCQQDYSSPLTFGAGTKLEIKRKSTAQL

SEQ ID NO: 3 - DNA, 478 bp, Artificial Sequence
  SYNTHESIZED
actggtgtgcagtcatgggcagacttacattctcattcctgctgctgctgcctgtccctgcatatgtcctatctcaggttactctgaaagagtctggccctgggatattgcagccatcacagacgctgagcctggcctgtactttctctgggatttcactgagtacttctggtttgggtttgagctggcttcgtaagccctcagggaaggctttagagtggctggtaagcatttggcctaatgaaaactacttcaacccatctttgaagagccggctcacaatctccaaggtgacctccaacaaccaagtattccttgaactcaccagtgtggacactgcagattctgccacgtactactgtgtttggagagagaagtgggagggtcactacgaatggtttgcttactggggccaagggactctggtcaccgtctctccagctagcacgacagccccatcggtcaagggcgaattcgc

SEQ ID NO: 4 - protein, 158 aa, Artificial Sequence
  SYNTHESIZED
WCAVMGRLTFSFLLLLPVPAYVLSQVTLKESGPGILQPSQTLSLACTFSGISLSTSGLGLSWLRKPSGKALEWLVSIWPNENYFNPSLKSRLTISKVTSNNQVFLELTSVDTADSATYYCVWREKWEGHYEWFAYWGQGTLVTVSPASTTAPSVKGEF

(19) United States
(12) Patent Application Publication
(10) Pub. No.: US 2020/0209263 A1
(43) Pub. Date: Jul. 2, 2020

(54) INCOMPATIBLE BLOOD GROUP ANTIGEN FOR CANCER DETECTION AND TREATMENT

(71) Applicant: Washington University, St. Louis, MO (US)

(72) Inventor: Mai Xu, St. Louis, MO (US)

(21) Appl. No.: 16/435,111

(22) Filed: Jun. 7, 2019

Related U.S. Application Data
(63) Continuation-in-part of application No. PCT/US2017/065382, filed on Dec. 8, 2017.
(60) Provisional application No. 62/431,795, filed on Dec. 8, 2016.

(51) Int. Cl. G01N 33/80; C12N 5/0783; A61K 47/68; A61K 49/00

(52) U.S. Cl. CPC G01N 33/80; C12N 5/0636; A61K 2123/00; A61K 49/0058; G01N 2800/24; A61K 47/6801

(57) ABSTRACT

The present invention provides compositions and methods directed to incompatible blood group antigens. In particular, the present invention relates to anti-incompatible BG-A antibody molecules targeting a novel immune epitope in the incompatible BG-A antigen, such as the epitope bound by CRC-A1. The invention also relates to nucleic acids encoding such antibody molecules; to host cells expressing or capable of expressing such antibody molecules; to compositions comprising such antibody molecules or fragments thereof; and to uses of such antibody molecules or such compositions, in particular for therapeutic and detection purposes in the field of cancer diseases.

### CROSS REFERENCE TO RELATED APPLICATIONS

This application claims the benefit of and is related to U.S. Provisional Application Ser. No. 62/431,795 filed on Dec. 8, 2016, which is hereby incorporated by reference in its entirety.

### FIELD OF THE INVENTION

The present invention provides compositions and methods directed to incompatible blood group antigens for therapeutic and detection purposes in the field of cancer diseases.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821(f).

### BACKGROUND OF THE INVENTION

In the United States, approximately 1.7 million new cases of cancer are diagnosed each year and there are about 15 million solid tumor survivors who require routine surveillance to monitor their tumor recurrence and treatment response. Approximately 154,000 of the newly diagnosed cases of cancer are colorectal cancer. Among them, an estimated 92,400 colorectal cancer patients are with blood type B and O. In addition to the newly diagnosed cases and estimated 750,000 colorectal cancer survivors with blood type B and O based on ABH blood type distribution in American. In addition, approximately 60 million people with blood type B and O aged 50 years and over are at high risk for colorectal cancer and are highly recommended to get a colonoscopy done for detection of early stage colorectal cancer. Colonoscopy screening for colorectal cancer alone costs healthcare industry about 1.2 billion dollars annually.

Approximately 50% of colorectal cancer patients are diagnosed at late stages of the disease. The five-year survival rate of patients with late stage disease is 11%. In contrast, the five-year survival rate for patients diagnosed at earlier stages (no metastasis) is 91%. Unfortunately, there are no sensitive, specific, non-invasive and cost-effective blood tests available for early detection and diagnosis of colorectal cancer. For example, the specificity and sensitivity rates of a carcinoembryonic antigen (CEA) blood test are 42% and 40-74% respectively. For the carbohydrate antigen 19-9 (CA19-9) blood test (also called cancer antigen 19-9 or sialylated Lewis (a) antigen), the specific and sensitive rates for colorectal cancer are 35% and 70%. The fecal occult blood test has variable specificity (65-98%) and sensitivity (73%). While more expensive methods CT and colonoscopy have higher specificity (90% and 94%), they also have lower sensitivity, 59-92% and 45-80% respectively, in addition they can be more invasive to the patients. Moreover, those methods are not suitable for routinely monitoring tumor recurrence and estimation of tumor response to therapeutic treatment.

Thus, there remains a need in the art for an accurate, none invasive, cost effective and personalized blood test for colorectal cancer detection and diagnosis.

### SUMMARY OF THE INVENTION

The present disclosure relates to compositions and methods relating to incompatible blood group antigens for therapeutics and detection of cancer.

According to one first aspect there are provided anti-incompatible BG-A antibody molecules. As described further herein, the anti-incompatible BG-A antibody molecules of the present invention bind to a novel epitope within the incompatible BG-A molecules. The binding epitope of antibodies of the present invention are different from those of BG-A antibodies naturally occurring in BG-B and BG-O serum, see for example FIG. 8.

Nucleic acid molecules encoding the anti-incompatible BG-A antibody molecules, expression vectors, host cells and methods of making the anti-incompatible BG-A antibody molecules of the invention are also provided. Pharmaceutical compositions comprising the anti-incompatible BG-A antibody molecules of the invention are also provided. The anti-incompatible BG-A antibody molecules disclosed herein, including bispecific antibodies and antibody drug conjugates, can be used to treat cancerous disorders, including solid and soft-tissue tumors.

In one aspect of the invention provides methods of using an anti-incompatible BG-A of the invention for detecting, monitoring progression or treatment of an incompatible BG-A antigen expressing cancer cell in a subject.

One aspect of the present disclosure encompasses T cells comprising a chimeric antigen receptor, wherein the chimeric antigen receptor specifically binds to an incompatible BG-A antigen.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 shows immunofluorescence staining of mAb CRC-A1 in human colon adenocarcinoma NSY cells. NSY cells (left) were unfixed and most of them were intensely stained with mAb CRC-A1. Internalization of mAb CRC-A1 was seen in the cultured cells after incubation for 40 min at 37° C. Cells in the right panel were fixed with 4% paraformaldehyde and 0.2% Triton X-100 and intensely stained on the cell membranes. Top, dark-field images. Bottom, bright-field images that served as controls.

FIG. 2 shows mAb CRC-A1 intensively stains colon cancer cells with various differentiation and histological types. Tissue microarray (TMA) analysis shown that positive rate of mAb CRC-A1 staining is 99% (N=189 with unknown ABO blood phenotype).

FIG. 3 depicts binding epitope analysis of mAb CRC-A1 on an antigen array that contains about 200 different glycans. Cy3-BSA was used as a positive control. mAb CRC-A1 binds to both BG-A and BG-A1.

FIG. 4 depicts representative images of immunochistochemical staining of mAb CRC-A1 in colorectal cancer tissue from patients with blood type B. The positive rate is 100% (N=16).

FIG. 5 shows representative images of immunohistochemical staining of mAb CRC-A1 in colorectal tissue from patients with blood type 0. The positive staining rate is 92% (N=25).

FIG. 6 shows immunohistochemical staining of mAb CRC-A1 in colon pre-cancerous tissues (adenoma) from patients with blood type B (upper panels) or O (lower panels). The pre-cancerous lesions were intensively stained with mAb CRC-A1.

FIG. 7A, FIG. 7B, and FIG. 7C show dual immunofluorescence staining of mAb CRC-A1 labeled with Alexa Fluor 488 (green) and Mab 293C3 to CD133 conjugated with PE dye (red) in human colon cancer differentiated (CD133−) and stem like (CD133+) cells isolated from NSY 42129 (FIG. 7A) and HT29 colon cancer cells (FIG. 7B). mAb CRC-A1 stained both colon cancer differentiated and stem like cells. In contrast, mAb CRC-A1 did not bound to PBMCs and CD34 positive cells (FIG. 7C).

FIG. 8 shows NSY42129 colon cancer cells and human colon cancer tissues were stained with mAb CRC-A1 after blocking with BG-B and BG-O serum containing anti-BG-A antibodies (mainly IgM). This figure indicates that the binding epitope of mAb CRC-A1 is different from those of BG-A antibodies naturally occurring in BG-B and BG-O serum.

FIG. 9 depicts optical imaging of a mouse with human colorectal xenograft tumor. Left panel demonstrates that CRC-A1-NIR probes specifically accumulated in tumor at 24 hours post injection. Right panel: Ex vivo images, the Mab CRC-A1 conjugates mainly maintained in tumor tissues at 120 hours post injection. Arrow head: liver; Arrow: tumor.

FIG. 10A, FIG. 10B, FIG. 10C and FIG. 10D show flowcytometry analysis of Mab CRC-A1 in the staining of acute myeloid leukemia (AML) cells from patient with blood type B. This figure show that Mab CRC-A1 reacts to AML cells from patients with Blood type B (FIG. 10C and FIG. 10D).

FIG. 11 shows bispecific T cell engagers (BiTE) construct of Mab CRC-A1 (left part of this figure) and T-cell receptors bind to antigens on the surface of tumor cells (right part).

FIG. 12 shows the develop immunotherapeutic reagents with mAb CRC-A1 for the treatment of cancer. Structure of Chimeric Antigen Receptor (CAR) with mAb CRC-A1 single-chain variable fragment (scFV). mAb CRC-A1 scFV: binding to a tumor specific epitope carried by incompatible BG-A; CD28: activating T cells; 4-1 BB: stimulatory ligands enhance T-cell proliferation; CD3: T cell receptor.

FIG. 13 depicts adoptive cell transfer therapy with CAR-engineered T cells (https://en.wikipedia.org/wiki/Chimeric_antigen_receptor).

FIG. 14A and FIG. 14B depicts the sequences of the CRC-A1 light and heavy chains. FIG. 14A shows the CRC-A1 light chain nucleotide and protein sequence as well as the corresponding CDRs. FIG. 14B shows the CRC-A1 heavy chain nucleotide and protein sequence as well as the corresponding CDRs (SEQ ID Nos: 1-4).

### DETAILED DESCRIPTION OF THE INVENTION

Applicants have discovered a truly specific biomarker (immune-epitope) for cancer detection and diagnosis, estimation of therapeutic efficacy, and monitoring of tumor progression. As described herein an immune-epitope carried by incompatible blood group A (BG-A) antigen provides a strictly tumor specific biomarker for cancer patients with blood type B and O. The ABO blood group system is used to denote the presence of one, both, or neither of the A and B antigens on erythrocytes. The BG-A antigen expressed in tumor cells from cancer patients with blood type O or blood type B is called incompatible BG-A antigen. The applicants have generated a monoclonal antibody designated as mAb CRC-A1, which selectively targets a novel epitope on the incompatible BG-A antigen expressed by cancer stem cells and differentiated cancer cells. The immune-epitope carried by incompatible blood group A (BG-A) antigen and targeted by the compositions of the invention provide 100% tumor specificity and between 80%-100% sensitivity.

Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Roitt et al., “Immunology” (2.sup.nd Ed.), Gower Medical Publishing, London, N.Y. (1989), as well as to the general background art cited herein. Furthermore, unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

### I. Compositions

Compositions of the disclosure are directed to compositions comprising incompatible BG-A antibodies or fragments thereof. Compositions of the disclosure are also directed to compositions comprising said antibodies as immunotherapeutic compositions. Various aspects of the disclosure will be described in further detail below.

### (a) Antibodies

In an aspect, the present disclosure provides an antibody which bind to a novel epitope presented by incompatible BG-A molecules. In one aspect, the antibody is generated by fusion of mouse myeloma P3X63Ag8.653 cells with B lymphocytes from a mouse immunized with colorectal cancer NSY 42129 cells as described herein. In one embodiment, mAb CRC-A1 antibody binds to an epitope within or presented by incompatible BG-A molecules. Methods of generating an antibody to an antigen are well known in the art. For example, monoclonal antibodies may be generated using a suitable hybridoma as would be readily understood by those of ordinary skill in the art. In the preferred process, a protein in accordance with the disclosure is first identified and isolated. Next, the protein is isolated and/or purified in any of a number of suitable ways commonly known in the art, or after the protein is sequenced, the protein used in the monoclonal process may be produced by recombinant means as would be commonly used in the art and then purified for use. In one suitable process, monoclonal antibodies may be generated from proteins isolated and purified as described above by mixing the protein with an adjuvant, and injecting the mixture into a laboratory animal. Immunization protocols may consist of a first injection (using complete Freund's adjuvant), two subsequent booster injections (with incomplete Freund's adjuvant) at three-week intervals, and one final booster injection without adjuvant three days prior to fusion. For hybridoma production, the laboratory animal may be sacrificed and their spleen removed aseptically. Antibody secreting cells may be isolated and mixed with myeloma cells (NS1) using drop-wise addition of polyethylene glycol. After the fusion, cells may be diluted in selective medium (vitamin-supplemented DMEM/HAT) and plated at low densities in 96 well plates. Tissue supernatants from the resulting fusion may be screened by ELISA, immunoblot techniques and tissue microarrays (TMAs). Cells from these positive wells may be grown and single cell cloned by limiting dilution, and supernatants subjected to one more round of screening by both ELISA and immunoblot. Positive clones may be identified, and monoclonal antibodies collected as hybridoma supernatants.

Anti-BG-A antibodies useful herein include all antibodies that specifically bind an epitope within an incompatible BG-A antigen. Specifically, the anti-BG-A antibodies may specifically bind an epitope present in the BG-A antigen, as described Dean L. Bethesda (Md.): National Center for Biotechnology Information (US); 2005. Chapter 5, herein incorporated by reference. In preferred embodiments, the anti-BG-A antibodies useful herein include antibodies that bind the epitope bound by mAb CRC-A1.

The term “antibody’ includes the term “monoclonal antibody”. “Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using e.g., hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

Further by “antibody” is meant a functional monoclonal antibody, or an immunologically effective fragment thereof; such as an Fab, Fab′, or F(ab′)2 fragment thereof. In some contexts herein, fragments will be mentioned specifically for emphasis; nevertheless, it will be understood that regardless of whether fragments are specified, the term “antibody” includes such fragments as well as single-chain forms. As long as the protein retains the ability specifically to bind its intended target, it is included within the term “antibody.” Also included within the definition “antibody” for example are single chain forms, generally designated Fv, regions, of antibodies with this specificity.

In particular, antibody fragments include, but are not limited to, (i) the Fab fragment consisting of VL, VH, CL and CH1 domains, including Fab and Fab—SH, (ii) the Fd fragment consisting of the VH and CH1 domains, (iii) the Fv fragment consisting of the VL and VH domains of a single antibody; (iv) the dAb fragment (Ward E S et al., (1989) Nature, 341: 544-546) which consists of a single variable, (v) F(ab′).sub.2 fragments, a bivalent fragment comprising two linked Fab fragments (vi) single chain Fv molecules (scFv), wherein a VH domain and a VL domain are linked by a peptide linker which allows the two domains to associate to form an antigen binding site (Bird R E et al., (1988) Science 242: 423-426; Huston J S et al., (1988) Proc. Natl. Acad. Sci. USA, 85: 5879-83), (vii) bispecific single chain Fv dimers (PCT/US92/09965), (viii) “diabodies” or “triabodies”, multivalent or multispecific fragments constructed by gene fusion (Tomlinson I & Hollinger P (2000) Methods Enzymol. 326: 461-79; WO94/13804; Holliger P et al., (1993) Proc Natl Acad Sci USA, 90: 6444-48) and (ix) scFv genetically fused to the same or a different antibody (Coloma M J & Morrison S L (1997) Nature Biotech, 15(2): 159-163).

Preferably, but not necessarily, the antibodies useful in the discovery are produced recombinantly, as manipulation of the typically murine or other non-human antibodies with the appropriate specificity is required in order to convert them to humanized form. Antibodies may or may not be glycosylated, though glycosylated antibodies are preferred. Antibodies are properly cross-linked via disulfide bonds, as is known.

The basic antibody structural unit of an antibody useful herein comprises a tetramer. Each tetramer is composed of two identical pairs of polypeptide chains, each pair having one “light’ (about 25 kDa) and one “heavy” chain (about 50-70 kDa). The amino-terminal portion of each chain includes a variable region of about 100 to 110 or more amino acid sequences primarily responsible for antigen recognition. The carboxy-terminal portion of each chain defines a constant region primarily responsible for effector function.

Light chains are classified as gamma, mu, alpha, and lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acid sequences, with the heavy chain also including a “D” region of about 10 more amino acid sequences.

The variable regions of each light/heavy chain pair form the antibody binding site. Thus, an intact antibody has two binding sites. The chains exhibit the same general structure of relatively conserved framework regions (FR) joined by three hypervariable regions, also called complementarity determining regions (hereinafter referred to as “CDRs.”) The CDRs from the two chains are aligned by the framework regions, enabling binding to a specific epitope. From N-terminal to C-terminal, both light and heavy chains comprise the domains FR1, CDR1, FR2, CDR2, FR3, CDR3 and FR4 respectively. The assignment of amino acid sequences to each domain is in accordance with known conventions (See, Kabat “Sequences of Proteins of Immunological Interest” National Institutes of Health, Bethesda, Md., 1987 and 1991; Chothia, et al, J. Mol. Bio. (1987) 196:901-917; Chothia, et al., Nature (1989) 342:878-883).

In an aspect, monoclonal anti-BG-A antibodies are generated with appropriate specificity by standard techniques of immunization of mammals, forming hybridomas from the antibody-producing cells of said mammals or otherwise immortalizing them, and culturing the hybridomas or immortalized cells to assess them for the appropriate specificity. In the present case, such antibodies could be generated by immunizing a, rabbit, rat or mouse, for example, with a peptide representing an epitope encompassing a region of the incompatible BG-A antigen or an appropriate subregion thereof. Materials for recombinant manipulation can be obtained by retrieving the nucleotide sequences encoding the desired antibody from the hybridoma or other cell that produces it. These nucleotide sequences can then be manipulated and isolated, characterized, purified and, recovered to provide them in humanized form, for use herein if desired.

As used herein “humanized antibody” includes an anti-BG-A antibody that is composed partially or fully of amino acid sequences derived from a human antibody germline by altering the sequence of an antibody having non-human complementarity determining regions (“CDR”). The simplest such alteration may consist simply of substituting the constant region of a human antibody for the murine constant region, thus resulting in a human/murine chimera which may have sufficiently low immunogenicity to be acceptable for pharmaceutical use. Preferably, however, the variable region of the antibody and even the CDR is also humanized by techniques that are by now well known in the art. The framework regions of the variable regions are substituted by the corresponding human framework regions leaving the non-human CDR substantially intact, or even replacing the CDR with sequences derived from a human genome. CDRs may also be randomly mutated such that binding activity and affinity for an incompatible BG-A antigen is maintained or enhanced in the context of fully human germline framework regions or framework regions that are substantially human. Substantially human frameworks have at least 90%, 95%, or 99% sequence identity with a known human framework sequence. Fully useful human antibodies are produced in genetically modified mice whose immune systems have been altered to correspond to human immune systems. As mentioned above, it is sufficient for use in the methods of this discovery, to employ an immunologically specific fragment of the antibody, including fragments representing single chain forms.

Further, as used herein the term “humanized antibody” refers to an anti-BG-A antibody comprising a human framework, at least one CDR from a nonhuman antibody, and in which any constant region present is substantially identical to a human immunoglobulin constant region, i.e., at least about 85-90%, preferably at least 95% identical. Hence, all parts of a humanized antibody, except possibly the CDRs, are substantially identical to corresponding pairs of one or more native human immunoglobulin sequences.

If desired, the design of humanized immunoglobulins may be carried out as follows. When an amino acid sequence falls under the following category, the framework amino acid sequence of a human immunoglobulin to be used (acceptor immunoglobulin) is replaced by a framework amino acid sequence from a CDR-providing nonhuman immunoglobulin (donor immunoglobulin): (a) the amino acid sequence in the human framework region of the acceptor immunoglobulin is unusual for human immunoglobulin at that position, whereas the corresponding amino acid sequence in the donor immunoglobulin is typical for human immunoglobulin at that position; (b) the position of the amino acid sequence is immediately adjacent to one of the CDRs; or (c) any side chain atom of a framework amino acid sequence is within about 5-6 angstroms (center-to-center) of any atom of a CDR amino acid sequence in a three dimensional immunoglobulin model (Queen, et al., op. cit., and Co, et al, Proc. Natl. Acad. Sci. USA (1991) 88:2869). When each of the amino acid sequences in the human framework region of the acceptor immunoglobulin and a corresponding amino acid sequence in the donor immunoglobulin is unusual for human immunoglobulin at that position, such an amino acid sequence is replaced by an amino acid sequence typical for human immunoglobulin at that position.

In all instances, an antibody of the disclosure specifically binds BG-A antigen. The phrase “specifically binds” herein means antibodies bind to the glycoprotein or glycolipids with an affinity constant or Affinity of interaction (KD) in the range of at least 0.1 mM to 1 pM, or in the range of at least 0.1 pM to 10 nM, with a preferred range being 0.1 pM to 1 nM. Methods of determining whether an antibody binds to a BG-A antigen are known in the art.

The antibodies of the present disclosure may also be used as fusion proteins known as single chain variable fragments (scFv). These scFvs are comprised of the heavy and light chain variable regions connected by a linker. In most instances, but not all, the linker may be a peptide. A linker peptide is preferably from about 10 to 25 amino acids in length. Preferably, a linker peptide is rich in glycine, as well as serine or threonine. ScFvs can be used to facilitate phage display or can be used for flow cytometry, immunohistochemistry, or as targeting domains. Methods of making and using scFvs are known in the art.

In a preferred embodiment, the scFvs of the present disclosure are conjugated to a human constant domain. In some embodiments, the heavy constant domain is derived from an IgG domain, such as IgG1, IgG2, IgG3, or IgG4. In other embodiments, the heavy chain constant domain may be derived from IgA, IgM, or IgE.

In one embodiment, an antibody of the disclosure may be derived from the mAb CRC-A1, and may comprise an amino acid sequence with 90%, 91%, 92%, 93%, 94%, 95%, 96% 97%, 98%, or 99% identity to the heavy chain variable region of SEQ ID NO:4 (WCAVMGRLTFSFLLLLPVPAYVLSQVTLKESGPGILQPSQTLSLACTFSGISLSTSGLG LSWLRKPSGKALEWLVSIWPNENYFNPSLKSRLTISKVTSNNQVFLELTSVDTADSATY YCVWREKWEGHYEWFAYWGQGTLVTVSPASTTAPSVKGEF), and/or may comprise an amino acid sequence with 90%, 91%, 92%, 93%, 94%, 95%, 96%, 97%, 98%, or 99% identity to the light chain variable region of SEQ ID NO:2 (IHQAGKGIKMKSQTQVFVFLLLCVSGAHGSIVMTQTPKFLLVSAGDRITITCKASQSVN NDVAWYQQKPGQSPKLLIYYASNRYTGVPDRFTGSGYGTDFTFTITTVKAEDPAVYFC QQDYSSPLTFGAGTKLEIKRKSTAQL). In an exemplary embodiment, an antibody of the disclosure that binds to BG-A antigen comprises the heavy chain amino acid sequence of SEQ ID NO:4 and the light chain amino acid sequence of SEQ ID NO:2. In one embodiment, an antibody of the disclosure is the monoclonal antibody referred to as CRC-A1. In one aspect, the antibody comprises one or more CDRs from the heavy chain variable region comprising SEQ ID NO:4 and one or more CDRs from the light chain variable region comprising SEQ ID NO:2.

In one embodiment, an antibody light chain of the disclosure may be derived from SEQ ID NO:1, or derived from a nucleotide which comprise a nucleotide sequence with 90%, 91%, 92%, 93%, 94%, 95%, 96% 97%, 98%, or 99% identity to SEQ ID NO:1 (GAATACATCAGGCAGGCAAGGGCATCAAGATGAAGTCACAGACCCAGGTCTTCGT ATTTCTACTGCTCTGTGTGTCTGGTGCTCATGGGAGTATTGTGATGACCCAGACTC CCAAATTCCTGCTTGTCTCAGCAGGAGACAGGATTACCATAACCTGCAAGGCCAGT CAGAGTGTGAATAATGATGTAGCTTGGTACCAACAGAAGCCAGGGCAGTCTCCTAA ACTGCTGATATACTATGCATCCAATCGCTACACTGGAGTCCCTGATCGCTTCACTG GCAGTGGATATGGGACGGATTTCACTTTCACCATCACCACTGTGAAGGCTGAAGA CCCGGCAGTTTATTTCTGTCAGCAGGATTATAGCTCTCCGCTCACGTTCGGTGCTG GGACCAAGCTGGAAATCAAACGTAAGTCGACTGCACAACTG), and/or may comprise a nucleotide sequence with 90%, 91%, 92%, 93%, 94%, 95%, 96%, 97%, 98%, or 99% identity to SEQ ID NO:3 (GAATACATCAGGCAGGCAAGGGCATCAAGATGAAGTCACAGACCCAGGTCTTCGT ATTTCTACTGCTCTGTGTGTCTGGTGCTCATGGGAGTATTGTGATGACCCAGACTC CCAAATTCCTGCTTGTCTCAGCAGGAGACAGGATTACCATAACCTGCAAGGCCAGT CAGAGTGTGAATAATGATGTAGCTTGGTACCAACAGAAGCCAGGGCAGTCTCCTAA ACTGCTGATATACTATGCATCCAATCGCTACACTGGAGTCCCTGATCGCTTCACTG GCAGTGGATATGGGACGGATTTCACTTTCACCATCACCACTGTGAAGGCTGAAGA CCCGGCAGTTTATTTCTGTCAGCAGGATTATAGCTCTCCGCTCACGTTCGGTGCTG GGACCAAGCTGGAAATCAAACGTAAGTCGACTGCACAACTG). In an exemplary embodiment, an antibody of the disclosure that binds to BG-A antigen comprises the heavy chain amino acid sequence and the light chain amino acid sequence or one or more CDRs from the heavy chain variable region and one or more CDRs from the light chain variable region resulting from the amino acid product of SEQ ID NO: 1 and SEQ ID NO:3.

The present disclosure is also directed to a host cell with a vector comprising the antibodies according to the present disclosure. The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Host cells include progeny of a single host cell, and the progeny may not necessarily be completely identical (in morphology or in total DNA complement) to the original parent cell due to natural, accidental, or deliberate mutation and/or change. A host cell includes a cell transfected or infected in vivo or in vitro with a recombinant vector or a polynucleotide of the present disclosure. A host cell, which comprises a recombinant vector of the invention, for example comprising SEQ ID NO: 1 and/or SEQ ID NO:3 or parts thereof, may also be referred to as a “recombinant host cell”.

The term “host cell(s)” refers to cell(s), which may be used in a process for purifying an immunogenic protein or recombinant antibody in accordance with the present disclosure. Such host cells carry the protein of interest (P01). A host cell may also be referred to as a protein-expressing cell. A host cell, according to the present invention, may be, but is not limited to, prokaryotic cells, eukaryotic cells, archeobacteria, bacterial cells, insect cells, yeast, mammal cells, and/or plant cells. Bacteria envisioned as host cells can be either gram-negative or gram-positive, e.g. Escherichia coli, Erwinia sp., Klebsellia sp., Lactobacillus sp. or Bacillus subtilis. Typical yeast host cells are selected from the group consisting of Saccharomyces cerevisiae, Hansenula polymorpha and Pichia pastoris.

To express a recombinant antibody according to the present disclosure, a DNA encoding a recombinant antibody or parts thereof, may be inserted into an expression vector such that the gene is operably linked to transcriptional and translational control sequences. In this context, the term “operably linked” means that a protein gene is ligated into a vector such that transcriptional and translational control sequences within the vector serve their intended function of regulating the transcription and translation of the protein gene. The expression vector and expression control sequences are chosen to be compatible with the expression host cell used. The isolated protein domain sequences are typically inserted into the same expression vector. The protein genes are inserted into the expression vector by standard methods. Additionally, the recombinant expression vector can encode a signal peptide that facilitates co-translational translocation of the nascent polypeptide chain into the endoplasmic reticulum (ER). The folded polypeptide (recombinant fusion protein according to this disclosure) may be secreted from a host cell or may be retained within the host cell. Intracellular retention or targeting can be achieved by the use of an appropriate targeting peptide such as C-terminal KDEL-tag for ER retrieval.

In general, those skilled in the art are well able to construct vectors and design protocols for recombinant gene expression. For further details see, for example, Molecular Cloning: a Laboratory Manual: 2nd edition, Sambrook et al, 1989, Cold Spring Harbor Laboratory Press (or later editions of this work) and Current Protocols in Molecular Biology, Second Edition, Ausubel et al. eds., John Wiley & Sons, 1992, which are incorporated herein by reference.

The above-described antibodies and their antigen binding portions can be used to make antibody drug conjugates. The composition described above can contain one or more antibody drug conjugates. An “antibody-drug conjugate” or “ADC” is an antibody that is conjugated to one or more (e.g., 1 to 4) cytotoxic agents or cytotoxins, e.g., through a linker or other means. As disclosed herein, the antibody can be a monoclonal antibody specific to a cancer antigen.

The antibody drug conjugate of this invention can be used as an anticancer therapeutic for all epithelial tumors such as breast, prostate, colorectal, esophageal, head/neck, skin, lung, tongue, cervical, stomach, ovary, pancreas and liver cancers as well as blood cancers (multiple myeloma and B-cell lymphomas including Burkitt's and Diffuse Large B-cell Lymphoma). This conjugate also can be used in the treatment of some tumors such as melanoma and glioma that are not of epithelial origin due to their expression of matriptase.

Various cytotoxic agents can be used. The term cytotoxic agent refers to a substance that inhibits or prevents the function of cells and/or causes destruction of cells. Examples include chemotherapeutic agents, such as methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. The term also encompasses compounds comprising one or more radioactive isotopes (e.g., At211, I131, I125 Y90, Re186, Re188, Sm153, Bi212, and P32.

To prepare ADCs, linker-cytotoxin conjugates can be made by convention methods analogous to those described in of Doronina et al., Bioconjugate Chem. 2006, 17, 114-124. Antibodies, typically monoclonal antibodies are raised against a specific cancer target antigen (e.g., activated matriptase), and purified and characterized. Therapeutic ADCs containing that antibody can be prepared by standard methods for cysteine conjugation, such as by methods analogous to that described in Hamblett et al., Clin. Cancer Res. 2004, 10, 7063-7070; Doronina et al., Nat. Biotechnol., 2003, 21(7), 778-784; and Francisco et al., Blood, 2003, 102, 1458-1465.

Antibody-drug conjugates with multiple (e.g., four) drugs per antibody can be prepared by partial reduction of the antibody with an excess of a reducing reagent such as DTT or TCEP at 37° C. for 30 min, then the buffer exchanged by elution through SEPHADEX G-25 resin with 1 mM DTPA in DPBS. The eluent is diluted with further DPBS, and the thiol concentration of the antibody may be measured using 5,5′-dithiobis(2-nitrobenzoic acid) [Ellman's reagent]. An excess, for example 5-fold, of the linker-cytotoxin conjugate is added at 4° C. for 1 hr, and the conjugation reaction may be quenched by addition of a substantial excess, for example 20-fold, of cysteine. The resulting ADC mixture may be purified on SEPHADEX G-25 equilibrated in PBS to remove unreacted linker-cytotoxin conjugate, desalted if desired, and purified by size-exclusion chromatography. The resulting ADC may then be then sterile filtered, for example, through a 0.2 μm filter, and lyophilized if desired for storage.

The ADCs of this invention may be assayed for binding affinity to and specificity for the desired antigen by any of the methods conventionally used for the assay of antibodies; and they may be assayed for efficacy as anticancer agents by any of the methods conventionally used for the assay of cytostatic/cytotoxic agents, such as assays for potency against cell cultures, xenograft assays, and the like as described in the examples below. A person of ordinary skill in the art will have no difficulty in determining suitable assay techniques; from the results of those assays, in determining suitable doses to test in humans as anticancer agents, and, from the results of those tests, in determining suitable doses to use to treat cancers in humans.

An ADC of this invention, like the antibody of this invention, can be formulated as solutions for intravenous administration, or as lyophilized concentrates for reconstitution to prepare intravenous solutions (to be reconstituted, e.g., with normal saline, 5% dextrose, or similar isotonic solutions). They can be administered by intravenous injection or infusion. A person of ordinary skill in the art will have no difficulty in developing suitable formulations.

Certain aspects of the present invention can be used to diagnose and/or image a disease associated with incompatible BG-A antigen expression, for example, colorectal cancer. In this regard, an antibody or antibody fragment can be detectably labeled through the use of radioisotopes, affinity labels (e.g., biotin, avidin, etc.), fluorescent labels, paramagnetic atoms, etc. The detection of foci of such detectably labeled antibodies or antibody fragments might be indicative of a site of tumor development. In a preferred embodiment, this technique is done in a non-invasive manner through the use of magnetic imaging, fluorography, PET, etc. Such a diagnostic test may be employed in monitoring the success of treatment of diseases, where presence or absence of BG-A antigen-positive cells is a relevant indicator. Such imaging may also be employed during surgical resection comprising metastatic disease to guide said surgical resection.

Visualization and quantification of MAb biodistribution using PET requires a suitable positron-emitting radionuclide. 89Zr (t½=78.4 h), 1241 (t½=100.3 h), 64Cu (t½=12.7 h), 86Y (ti/2=14.7 h), and 76Br (t½=16.2 h) are well suited for imaging of intact MAbs.

Depending on the imaging agent or agents used, detecting the imaging agent can comprise performing fluorescence imaging, PET, MRI, CT, SPECT or a combination thereof on the subject. For example, in the case of NIR fluorescence imaging, images can be acquired using a fluorescence imaging system as described in U.S. Pat. Nos. 7,328,059 and 8,084,753 and U.S. Patent Publications US20080064954, US20100305453, US201 10071403, US2011028081 1, as well as in Sevick-Muraca 2012 (Translation of near-infrared fluorescence imaging technologies: emerging clinical applications. Annu. Rev. Med. 63, 217-231), each of which is incorporated herein by reference. In a further aspect, the imaging agent can be a fluorophore, a dye, an MRI contrast agent or a radionuclide. In certain aspects, a subject for imaging is a subject having as cancer. Accordingly, in aspects, imaging a subject comprises imaging cancer cells in a subject. For example, a method can be defined as a method for detecting cancer metastasis, such as lymphoid metastasis (e.g., lymph node metastasis of a prostate cancer) or for delineating tumor margins and infiltrating cancer within the surgical field of view. Antibody-imaging agent, conjugates can be made by convention methods known to those skilled in the art. Imagining agents for use according to the embodiments include, without limitation, fluorophores, dyes, a MRI active agent, radionuclides or a fluorescence radionuclide. In certain aspects, an antibody can be conjugated to two or more agents, such as to a fluorophore and a radio-imaging agent.

Antibody, therapeutics and imaging agents detailed herein may be administered systemically, locally or distally. In some aspects, an antibody may be administered intravenously, intradermally, intratumorally, intramuscularly, intraperitoneally or subcutaneously.

In an aspect, the present disclosure provides a bispecific antibody capable of binding to incompatible BG-A antigen. In preferred embodiments, the bispecific antibody binds to the CRC-A1 epitope on an incompatible BG-A antigen. A bispecific antibody comprising two antibody variable domains on a single polypeptide chain or by a synthetic polypeptide linker, wherein a first portion of the bispecific antibody is capable of recruiting the activity of a human immune effector cell by specifically binding to an effector antigen on the human immune effector cell, said first portion consisting of one antibody variable domain, and a second portion of the bispecific antibody specifically binds to a target antigen other than the effector antigen, for example incompatible BG-A antigen, said target antigen on a target cell other than said human immune effector cell, for example on a cancer cell, and said second portion comprising one antibody variable domain.

According to one embodiment, the bispecific antibody capable of binding to a BG-A antigen comprises a first portion wherein the first portion comprises a VH and/or VL variable region from mAb CRC-A1 and a second portion which is capable of recruiting the activity of a human immune effector cell. According to one embodiment, the bispecific antibody capable of binding to a BG-A antigen comprises a first portion wherein the first portion comprises a VH and/or VL variable region SEQ ID NO:2 and/or SEQ ID NO:4 and a second portion which is capable of recruiting the activity of a human immune effector cell.

The term “human immune effector cell” refers to a cell within the natural repertoire of cells in the human immune system which, when activated, is able to bring about a change in the viability of a target cell. The term “viability of a target cell” may refer within the scope of the invention to the target cell's ability to survive, proliferate and/or interact with other cells. Such interaction may be either direct, for example when the target cell contacts another cell, or indirect, for example when the target cell secretes substances which have an influence on the functioning of another distant cell. The target cell may be either native or foreign to humans. In the event that the cell is native to humans, the target cell is advantageously a cell which has undergone transformation to become a malignant cell. The native cell may additionally be a pathologically modified native cell, for example, a native cell infected with an organism such as a virus, a plasmodium or a bacterium. In the event that the cell is foreign to humans, the target cell is advantageously an invading pathogen, for example, an invading bacterium or plasmodium. According to a further embodiment of the invention, the antibody variable domains of the first and/or second portions may be derived from identical or separate animal species. This has the advantage that for each portion of the bispecific antibody, optimal antibody variable domain/s can be chosen to be derived from the animal species known to yield the best antibodies against a particular effector and/or target antigen. In this way, this embodiment allows the researcher to capitalize on already known, developed and/or optimized specificities such that the efficiency of workflow in developing bispecific antibodies as described herein is maximized.

In one preferred embodiment, the first and/or second portion of the bispecific antibody are/is independently derived from an antibody produced in primate, rodent, tylopoda or cartilaginous fish.

The first and/or second portion of a bispecific antibody according to this embodiment may be either naturally occurring or genetically engineered. Alternatively, it is within the scope of the present embodiment that part of a naturally occurring antibody is used as a substrate on which further genetic engineering is performed, to finally yield a derivative of the naturally occurring part of the antibody for use in the first or second portion of a bispecific antibody according to this embodiment.

In the event that the first and/or second portion of the bispecific antibody are/is derived from rodent, said first and/or second portion may advantageously be derived independently from mouse or rat antibodies. In this way, one seeking to develop and/or optimize bispecific antibodies according to this embodiment of the invention can benefit from the preexisting and highly diverse palette of known murine and rat antibody sequences which bind relevant human antigens.

In the event that a primate antibody is used as a basis for the first and/or second portion of the bispecific antibody, said first and/or second portion are/is advantageously derived independently from human antibodies. Besides benefiting from the ever-growing diversity of known human antibodies, use of human antibody variable domains entails the further advantage that the resulting bispecific antibodies will elicit little to no immunogenic response when administered as part of a therapeutic regimen in human patients. Such bispecific antibodies are thus especially suitable as therapeutic agents for use in humans. In the event that a tylopoda-derived antibody variable domain is used in the first and/or second portion of a bispecific antibody according to this embodiment of the invention, said first and/or second portion may advantageously be derived independently from camel, llama or/and dromedary. This use of such “camelid” antibodies allows the researcher seeking to develop or optimize bispecific antibodies according to this embodiment of the invention to capitalize on the unique types of antibodies known to be produced by these species. These species are namely known to produce high affinity antibodies of only a single variable domain. In the event that a tylopoda antibody is used as the source for the antibody variable domain in the first and/or second portion of the bispecific antibody, it is advantageous to use the NHH domain or a modified variant thereof.

The term “NHH” denotes a variable region of a heavy chain of a so-called “camelid” antibody. Camelid antibodies comprise a heavy chain, but lack a light chain. As such, a NHH region from such a camelid antibody represents the minimal structural element required to specifically bind to an antigen of interest in these species.

Camelid NHH domains have been found to bind to antigen with high affinity (Desmyter et al. 2001. J Biol Chem 276, 26285-90) and possess high stability in solution (Ewert et al. 2002. Biochemistry 41, 3628-36).

In the event that said first and/or second portion of the bispecific antibody is derived from a cartilaginous fish, said cartilaginous fish is advantageously a shark.

In the event that a rodent or primate antibody is used as the source for the antibody variable domain in the first and/or second portion of a bispecific antibody according to this embodiment of the invention, it is advantageous to use the NH domain or a modified variant thereof. The NH domain of antibodies in these species is known to contribute significantly to the binding specificity and affinity observed for a given antibody. At an absolute minimum, it is advantageous to use at least the third complementarity determining region (CDR) from a NH domain of such a parent antibody in designing the first and/or second portion of the bispecific antibody. This is due to the fact that the NH-CDR3 is known to play a major role in the specificity and affinity of binding of all the CDR regions, of which there are three in each of VH and VL. According to a further embodiment of the invention, the bispecific antibody may be subjected to an alteration to render it less immunogenic when administered to a human. Such an alteration may comprise one or more of the techniques commonly known as chimerization, humanization, CDR-grafting, deimmunization and/or mutation of framework region amino acids to correspond to the closest human germline sequence (germlining). Subjecting the bispecific antibody of the invention to such an alteration/s has the advantage that a bispecific antibody which would otherwise elicit a host immune response is rendered more, or completely “invisible” to the host immune system, so that such an immune response does not occur or is reduced. Bispecific antibodies which have been altered as described according to this embodiment will therefore remain administrable for a longer period of time with reduced or no immune response-related side effects than corresponding bispecific antibodies which have not undergone any such alteration(s). One of ordinary skill in the art will understand how to determine whether, and to what degree an antibody must be altered in order to prevent it from eliciting an unwanted host immune response.

According to another embodiment of the invention, the human immune effector cell is a member of the human lymphoid cell lineage, in this embodiment, the effector cell may advantageously be a human T cell, a human B cell or a human natural killer (NK) cell. Advantageously, such cells will have either a cytotoxic or an apoptotic effect on the target cell. Especially advantageously, the human lymphoid cell is a cytotoxic T cell which, when activated, exerts a cytotoxic effect on the target cell. According to this embodiment, then, the recruited activity of the human effector cell is this cell's cytotoxic activity.

According to a preferred embodiment, activation of the cytotoxic T cell may occur via binding of the CD3 antigen as effector antigen on the surface of the cytotoxic T cell by a bispecific antibody of this embodiment of the invention. The human CD3 antigen is present on both helper T cells and cytotoxic T cells. Human CD3 denotes an antigen which is expressed on T cells as part of the multimolecular T cell complex and which comprises three different chains: CD3-epsilon, CD3-delta and CD3-gamma.

The activation of the cytotoxic potential of T cells is a complex phenomenon which requires the interplay of multiple proteins. The T cell receptor (“TCR”) protein is a membrane bound disulfide-linked heterodimer consisting of two different glycoprotein subunits. The TCR recognizes and binds foreign peptidic antigen which itself has been bound by a member of the highly diverse class of major histocompatibility complex (“MHC”) proteins and has been presented, bound to the MHC, on the surface of antigen presenting cells (“APCs”).

Although the variable TCR binds foreign antigen as outlined above, signaling to the T cell that this binding has taken place depends on the presence of other, invariant, signaling proteins associated with the TCR. These signaling proteins in associated form are collectively refined to as the CD3 complex, here collectively refined to as the CD3 antigen.

The activation of T cell cytotoxicity, then, normally depends first on the binding of the TCR with an MHC protein, itself bound to foreign antigen, located on a separate cell. Only when this initial TCR-MHC binding has taken place can the CD3-dependent signaling cascade responsible for T cell clonal expansion and, ultimately, T cell cytotoxicity ensue.

However, binding of the human CD3 antigen by the first or second portion of a bispecific antibody of the invention activates T cells to exert a cytotoxic effect on other cells in the absence of independent TCR-MHC binding. This means that T cells may be cytotoxically activated in a clonally independent fashion, i.e. in a manner which is independent of the specific TCR clone carried by the T cell. This allows an activation of the entire T cell compartment rather than only specific T cells of a certain clonal identity.

In light of the foregoing discussion, then, an especially preferred embodiment of the invention provides a bispecific antibody in which the effector antigen is the human CD3 antigen. The bispecific antibody according to this embodiment of the invention may have a total of either two or three antibody variable domains.

According to further embodiments of the invention, other lymphoid cell-associated effector antigens bound by a bispecific antibody of the invention may be the human CD 16 antigen, the human NKG2D antigen, the human NKp46 antigen, the human CD2 antigen, the human CD28 antigen or the human CD25 antigen.

According to another embodiment of the invention, the human effector cell is a member of the human myeloid lineage. Advantageously, the effector cell may be a human monocyte, a human neutrophilic granulocyte or a human dendritic cell. Advantageously, such cells will have either a cytotoxic or an apoptotic effect on the target cell. Advantageous antigens within this embodiment which may be bound by a bispecific antibody of the invention may be the human CD64 antigen or the human CD89 antigen.

According to another embodiment of the invention, the target antigen is an antigen which is uniquely expressed on a target cell in a disease condition, but which remains non-expressed, expressed at a low level or non-accessible in a healthy condition. Examples of such target antigens which might be specifically bound by a bispecific antibody of the invention may advantageously be incompatible BG-A antigen. According to a preferred embodiment, the target antigen specifically bound by a bispecific antibody may be a cancer-related antigen that is an antigen related to a malignant condition. Such an antigen is either expressed or accessible on a malignant cell, whereas the antigen is either not present, not significantly present, or is not accessible on a non-malignant cell. As such, a bispecific antibody according to this embodiment of the invention is a bispecific antibody which recruits the activity of a human immune effector cell against the malignant target cell bearing the target antigen, or rendering the target antigen accessible.

In an especially preferred embodiment of the invention, the bispecific antibody specifically binds to the human CD3 antigen as effector antigen and to the BG-A antigen as target antigen. A bispecific antibody according to this embodiment consists of two or three antibody variable domains, separated by spacer and possibly linker polypeptides as described above.

Bispecific or heterodimeric antibodies have been available in the art for many years. However, the generation of such antibodies is often associated with the presence of mispaired by-products, which reduces significantly the production yield of the desired bispecific antibody and requires sophisticated purification procedures to achieve product homogeneity. The mispairing of immunoglobulin heavy chains can be reduced by using several rational design strategies, most of which engineer the antibody heavy chains for heterodimerisation via the design of man-made complementary heterodimeric interfaces between the two subunits of the CH3 domain homodimer. The first report of an engineered CH3 heterodimeric domain pair was made by Carter et al. describing a “protuberance-into-cavity” approach for generating a hetero-dimeric Fc moiety (U.S. Pat. No. 5,807,706; Inobs-into-holes'; Merchant A M et al., (1998) Nat Biotechnol, 16(7):677-81). Alternative designs have been recently developed and involved either the design of a new CH3 module pair by modifying the core composition of the modules as described in WO2007110205 or the design of complementary salt bridges between modules as described in WO2007147901 or WO2009089004. The disadvantage of the CH3 engineering strategies is that these techniques still result in the production of a significant number of undesirable homo-dimers. A more preferred technique for generating bispecific antibodies in which predominantly heterodimers are produced is described in PCT Publication No: WO2012/131555. Bispecific antibodies can be generated to a number of targets, for example, a target located on tumor cells and/or a target located on effector cells. Preferably, a bispecific antibody can bind to two targets selected from the group consisting of: AXL, Bcl2, BG-A, HER2, HERS, EGF, EGFR, VEGF, VEGFR, IGFR, PD-1, PD-1L, BTLA, CTLA-4, GITR, mTOR, CS1, CD3, CD16, CD16a, CD19, CD20, CD22, CD25, CD27, CD28, CD30, CD32b, CD33, CD38, CD40, CD52, CD64, CD79, CD89, CD137, CD138, CA125, cMet, CCR6, MUCI, PEM antigen, Ep-CAM, EphA2, 17-1a, CEA, AFP, HLA class II, HLA-DR, HSG, IgE, IL-12, IL-17a, IL-18, IL-23, IL-1alpha, IL-1beta, GD2-ganglioside, MCSP, NG2, SK-I antigen, Lag3, PAR2, PDGFR, PSMA, Tim3, TF, CTLA4, TL1A, TIGIT, SIRPa, ICOS, Treml2, NCR3, HVEM, OX40, VLA-2 and 4-1BB.

### (b) CAR T-Cells

One aspect of the present disclosure encompasses T cells comprising a chimeric antigen receptor, wherein the chimeric antigen receptor specifically binds to an incompatible BG-A antigen.

A CAR-T cell is a T cell that expresses a chimeric antigen receptor. The phrase “chimeric antigen receptor (CAR),” as used herein and generally used in the art, refers to a recombinant fusion protein that has an antigen-specific extracellular domain coupled to an intracellular domain that directs the cell to perform a specialized function upon binding of an antigen to the extracellular domain. The terms “artificial T-cell receptor,” “chimeric T-cell receptor,” and “chimeric immunoreceptor” may each be used interchangeably herein with the term “chimeric antigen receptor.” Chimeric antigen receptors are distinguished from other antigen binding agents by their ability to both bind MHC-independent antigen and transduce activation signals via their intracellular domain. The extracellular and intracellular portions of a CAR are discussed in more detail below.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (KD) between about 0.1 pM to about 10 μM, preferably about 0.1 pM to about 1 μM, more preferably about 0.1 pM to about 100 nM. Methods for determining the affinity of interaction are known in the art. An antigen-specific extracellular domain suitable for use in a CAR of the present disclosure is antigen-binding polypeptide comprising SEQ ID NO:2 and/or SEQ ID NO:4 or parts thereof. In one embodiment, a CAR-T cell of the present disclosure comprises an extracellular domain of a chimeric antigen receptor that specifically binds to the incompatible BG-A antigen. In some instances, the antigen-binding domain is a single chain Fv (scFv) comprising SEQ ID NO:2 and/or SEQ ID NO:4 or parts thereof, for example one or more CDRs. Other antibody based recognition domains (cAb VHH (camelid antibody variable domains) and humanized versions thereof, IgNAR VH (shark antibody variable domains) and humanized versions thereof, sdAb VH (single domain antibody variable domains) and “camelized” antibody variable domains are suitable for use. In some instances, T-cell receptor (TCR) based recognition domains such as single chain TCR (scTv, single chain two-domain TCR containing V.alpha.V.beta.) are also suitable for use.

A chimeric antigen receptor of the present disclosure also comprises an intracellular domain that provides an intracellular signal to the T cell upon antigen binding to the antigen-specific extracellular domain. The intracellular signaling domain of a chimeric antigen receptor of the present disclosure is responsible for activation of at least one of the effector functions of the T cell in which the chimeric receptor is expressed. The term “effector function” refers to a specialized function of a differentiated cell. An effector function of a T cell, for example, may be cytolytic activity or helper activity including the secretion of cytokines. An effector function in a naive, memory, or memory-type T cell may also include antigen-dependent proliferation. Thus, the term “intracellular domain” refers to the portion of a CAR that transduces the effector function signal upon binding of an antigen to the extracellular domain and directs the T cell to perform a specialized function. Non-limiting examples of suitable intracellular domains include the zeta chain of the T-cell receptor or any of its homologs (e.g., eta, delta, gamma, or epsilon), MB 1 chain, B29, Fc RIII, Fc RI, and combinations of signaling molecules, such as CD3.zeta. and CD28, CD27, 4-1 BB, DAP-10, OX40, and combinations thereof, as well as other similar molecules and fragments. Intracellular signaling portions of other members of the families of activating proteins may be used, such as Fc.gamma.RIII and Fc.epsilon.RI. While usually the entire intracellular domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term intracellular domain is thus meant to include any truncated portion of the intracellular domain sufficient to transduce the effector function signal.

Typically, the antigen-specific extracellular domain is linked to the intracellular domain of the chimeric antigen receptor by a transmembrane domain. A transmembrane domain traverses the cell membrane, anchors the CAR to the T cell surface, and connects the extracellular domain to the intracellular signaling domain, thus impacting expression of the CAR on the T cell surface. Chimeric antigen receptors may also further comprise one or more costimulatory domain and/or one or more spacer. A costimulatory domain is derived from the intracellular signaling domains of costimulatory proteins that enhance cytokine production, proliferation, cytotoxicity, and/or persistence in vivo. A spacer connects (i) the antigen-specific extracellular domain to the transmembrane domain, (ii) the transmembrane domain to a costimulatory domain, (iii) a costimulatory domain to the intracellular domain, and/or (iv) the transmembrane domain to the intracellular domain. For example, inclusion of a spacer domain between the antigen-specific extracellular domain and the transmembrane domain may affect flexibility of the antigen-binding domain and thereby CAR function. Suitable transmembrane domains, costimulatory domains, and spacers are known in the art.

Methods for CAR design, delivery and expression in T cells, and the manufacturing of clinical-grade CAR-T cell populations are known in the art. See, for example, Lee et al., Clin. Cancer Res., 2012, 18(10): 2780-90, hereby incorporated by reference in its entirety. For example, the engineered CARs may be introduced into T cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

CAR-T cells may be generated from any suitable source of T cells known in the art including, but not limited to, T cells collected from a subject. The subject may be a patient with a malignancy in need of CAR-T cell therapy or a subject of the same species as the subject with the malignancy in need of CAR-T cell therapy. The collected T cells may be expanded ex vivo using methods commonly known in the art before transduction with a CAR to generate a CAR-T cell.

The use of autologous T cells for the generation of CAR-T cells, while possible, may present unique challenges. The subjects in need of CAR-T cell therapy may be undergoing treatment for malignancies and this treatment may have affected the number and function of T cells of the host, thereby reducing the number of T cells that may be efficiently engineered into CAR-T cells. Also, T-cell hematologic malignancies and normal T effectors may co-express many of the same surface antigens making it very difficult to purify normal T effectors away from the malignant T cells for genetic editing and lentiviral transduction. Also, if the process of purification is not absolute, there may be a risk of deleting the target antigen such as CD7 in the malignant T cells resulting in the generation of a population of contaminating T cell cancers that are potentially resistant to the fratricide CAR-T cell. Thus, to avoid contamination risk of normal effector T cells with malignant T cell, the use of patient-derived T cells to generate CAR-T cells for T cell malignancies may not be desirable.

To overcome the contamination risk, T cells from another subject (a donor subject), without T cells malignancies may be used to generate CAR-T cells for allogeneic therapy. The T cells for allogeneic therapy may be collected from a single subject or multiple subjects. Methods of collecting blood cells, isolating and enriching T cells, and expanding them ex vivo may be by methods known in the art.

In an exemplary embodiment, the CAR for the incompatible BG-A antigen specific CAR T-cell may be generated by cloning a synthesized CRC-A1 single chain variable fragment (scFv) into a 3rd generation CAR backbone with CD28 and 4-1 BB internal signaling domains. An extracellular hCD34 domain may be added after a P2A peptide to enable both detection of CAR following viral transduction and purification using anti-hCD34 magnetic beads.

### (c) Pharmaceutical Compositions

The immunotheraputic composition or an antibody disclosed herein can be formulated into various dosage forms and administered by a number of different means that will deliver a therapeutically effective amount of the antigen or antibody. Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be an intramuscular formulation.

Solid dosage forms for oral administration include capsules, tablets, caplets, pills, powders, pellets, and granules. In such solid dosage forms, the active ingredient is ordinarily combined with one or more pharmaceutically acceptable excipients. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, an antigen or antibody of the disclosure is encapsulated in a suitable vehicle to either aid in the delivery of the antigen or antibody to target cells, to increase the stability of the composition, or to minimize potential toxicity of the composition. As will be appreciated by a skilled artisan, a variety of vehicles are suitable for delivering a composition of the present disclosure. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of antigen or antibody in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, antigen may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), phosphatidylethanolamine (PE), and linear polyethylenimine (I-PEI). In a specific embodiment, the liposome may be comprised of linear polyethylenimine (I-PEI). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which sphingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying antigen or antibody may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the disclosure may be delivered as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The antigen or antibody may be encapsulated in a microemulsion by any method generally known in the art.

In yet another embodiment, antigen or antibody may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the disclosure therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

### II. Methods

In an aspect, the present disclosure provides methods of detecting a cancer, monitoring tumor progression, and estimating therapeutic efficacy in treating the incompatible BG-A antigen expressing cancer in a subject. In a preferred embodiment, the subject of the above methods has type B or type O blood. The method comprises contacting a subject or a biological sample obtained from the subject with an anti-incompatible BG-A antibody or contacting with a plurality of CAR T-cells as disclosed herein or contacting the with a composition comprising a bispecific antibody as described herein. As used herein, “subject” or “patient” is used interchangeably.

An “effective dose” or “therapeutically effective amount” as used herein, means an amount which provides a therapeutic or prophylactic benefit.

The term “therapeutic effect” as used herein, refers to a biological effect which can be manifested by a decrease in the number of cancer cells, an increase in life expectancy, or amelioration of various physiological symptoms associated with the malignant condition, etc. For example, a therapeutically or pharmaceutically effective amount of a composition is the amount required to produce a desired therapeutic effect as may be judged by clinical trial results and/or model animal studies. The effective or pharmaceutically effective amount depends on several factors, including but not limited to, the route of administration, the stage of disease progression, characteristics of the subject (for example height, weight, sex, age and medical history), location of disease, the therapeutic composition used and/or the particular antibody used.

The concentration of anti-incompatible BG-A antibody in compositions to be administered is an effective amount and ranges from as low as about 0.1% by weight to as much as about 15 or about 20% by weight of the composition and will be selected primarily based on fluid volumes, viscosities, and so forth, in accordance with the particular mode of administration selected if desired. A typical composition comprising an anti-BG-A antibody for injection to a living subject could be made up to contain from 1-5 mL sterile buffered water or phosphate buffered saline and about 1-5000 mg of anti-incompatible BG-A antibody. A typical composition for intravenous infusion could have volumes between 1-250 mL of fluid, such as sterile Ringer's solution, and 1-100 mg per ml, or more of anti-incompatible BG-A antibody concentration. Doses will vary from subject to subject based on size, weight, and other physio-biological characteristics of the subject receiving the successful administration. In an aspect, a typical dose contains from about 0.01 mg/kg to about 100 mg/kg of an anti-BG-A antibody described herein. Doses can range from about 0.05 mg/kg to about 100 mg/kg, more preferably from about 0.1 mg/kg to about 50 mg/kg, or from 0.5 mg/kg to about 50 mg/kg, or from about 10 mg/kg to about 50 mg/kg. In a specific embodiment, the dose of anti-incompatible BG-A antibody may range from about 10 mg/kg to about 50 mg/kg.

Following initial administration of a composition of the disclosure, subjects may receive one or several additional administrations of the composition adequately spaced. Dosing treatment can be a single dose schedule or a multiple dose schedule. Suitable timing between doses (e.g. between 4-16 weeks) can be routinely determined.

A composition of the disclosure may be administered as multiple doses. In the treatment of a cancer, a composition of the disclosure may be administered as multiple doses. Administration may be daily, twice daily, weekly, twice weekly, monthly, twice monthly, every 6 weeks, every 3 months, every 6 months or yearly. For example, administration may be every 2 weeks, every 3 weeks every 4 weeks, every 5 weeks, every 6 weeks, every 7 weeks, every 8 weeks, every 9 weeks, every 10 weeks, every 11 weeks or every 12 weeks. Alternatively, administration may be every 1 month, every 2 months, every 3 months, every 4 months, every 5 months, every 6 months, every 7 months, every 8 months, every 9 months, every 10 months, every 11 months or every 12 months. Still further, administration may be every 1 year, every 2 years, every 3 years, every 4 years, every 5 years, every 6 years, every 7 years, every 8 years, every 9 years, every 10 years, every 15 years or every 20 years. The duration of treatment can and will vary depending on the subject and the cancer to be prevented or treated. For example, the duration of treatment may be for 1 week, 2 weeks, 3 weeks, 4 weeks, 5 weeks, 6 weeks. Alternatively, the duration of treatment may be for 1 month, 2 months, 3 months, 4 months, 5 months, 6 months, 7 months, 8 months, 9 months, 10 months, 11 months, or 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, administration may be every 4 weeks for 6 months to a year and then administration may be every year thereafter.

A method of the disclosure may further comprise administering an anticancer agent. As used herein, an “anticancer agent” is an agent that kills cancer cells or inhibits their growth. Anticancer agents are well known in the art.

Suitable subjects include, but are not limited to, a human, a livestock animal, a companion animal, a lab animal, and a zoological animal.

### (a) Methods of Using Anti-Incompatible BG-A Antibodies

In another aspect, the present invention encompasses methods for detecting an incompatible BG-A antigen expressing cancer cell in a subject. The method comprises (a) obtaining a biological sample from a subject, and (b) determining the presence or absence of the incompatible BG-A antigen with an anti-incompatible BG-A antibody of the disclosure, and optionally (c) identifying or diagnosing a subject as having a incompatible BG-A antigen expressing cancer cell when the antibody recognizes an incompatible BG-A antigen present in the biological sample. Alternatively, the method generally comprises (a) measuring the amount of the incompatible BG-A antigen in a biological sample obtained from a subject using an anti-incompatible BG-A antibody of the disclosure, (b) comparing the amount of the incompatible BG-A antigen in the sample to a reference value, and (c) classifying the subject as having a high or low amount of the incompatible BG-A antigen relative to the reference value based on the amount of the incompatible BG-A antigen measured in the sample. In a preferred embodiment, the biological sample is biological fluid selected from the group consisting of urine, sputum, saliva, ascites, pleural effusion, blood, plasma, and serum. In a more preferred embodiment, the amount of the incompatible BG-A antigen is measured in the exosomal fraction of a biological fluid of a subject with type B or type O blood.

As used herein, the term “exosome” refers to cell-derived vesicles that are present in many and perhaps all biological fluids, including blood, plasma, serum, urine, ascites, pleural effusion, saliva and cell culture supernatant. The reported diameter of exosomes is between 30 and 100 nm, which is larger than LDL, but much smaller than, for example red blood cells. Exosomes are either released from the cell when multivesicular bodies fuse with the plasma membrane or they are released directly from the plasma membrane. It is becoming increasingly clear that exosomes have specialized functions and play a key role in, for example, coagulation, intercellular signaling and waste management. Exosomes can potentially be used for prognosis, therapy, and biomarkers carried by exosomes for health and disease.

In certain aspects, the cancer is a breast cancer, lung cancer, head and neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer, ovarian, leukemia or skin cancer. In certain aspects, the cancer is a colorectal cancer. In one aspect, the subject has previously been treated for a cancer or has previously had a tumor surgically removed.

Immunohistochemical staining may be used to measure the differential expression of the incompatible BG-A antigen. This method enables the localization of a biomarker (antigen) in the cells of a tissue section by interaction of the antigen with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary anti-BG-A antibody as described herein, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of the incompatible BG-A antigen. There are many variations of an ELISA assay. All are based on the immobilization of an antigen or antibody on a solid surface, generally a microtiter plate. In one aspect, an entire exosome or exosome particles isolated from a biological sample is immobilized on a microtiter plate and an anti-incompatible BG-A antibody as described herein is used as a primary antibody (ExoELISA). The original ELISA method comprises preparing a sample containing the biomarker of interest, coating the wells of a microtiter plate with the sample, incubating each well with a primary antibody that recognizes a specific antigen or biomarker, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

As used herein, “obtaining a biological sample” or “obtaining a blood sample” refer to receiving a biological or blood sample, e.g., either directly or indirectly. For example, in some embodiments, the biological sample, such as a blood sample or a sample containing peripheral blood mononuclear cells (PBMC), is directly obtained from a subject at or near the laboratory or location where the biological sample will be analyzed. In other embodiments, the biological sample may be drawn or taken by a third party and then transferred, e.g., to a separate entity or location for analysis. In other embodiments, the sample may be obtained and tested in the same location using a point-of care test. In these embodiments, said obtaining refers to receiving the sample, e.g., from the patient, from a laboratory, from a doctor's office, from the mail, courier, or post office, etc. In some further aspects, the method may further comprise reporting the determination to the subject, a health care payer, an attending clinician, a pharmacist, a pharmacy benefits manager, or any person that the determination may be of interest.

As used herein, the term “biological sample” refers to a sample obtained from a subject. Numerous types of biological samples are known in the art. Suitable biological samples may include, but are not limited to, tissue samples or bodily fluids. In some embodiments, the biological sample is a tissue sample such as a tissue biopsy of tissues. The tissue biopsy may be a biopsy of a known or suspected cancer. The biopsied tissue may be fixed, embedded in paraffin or plastic, and sectioned, or the biopsied tissue may be frozen and cryosectioned. Alternatively, the biopsied tissue may be processed into individual cells or an explant, or processed into a homogenate, a cell extract, a membranous fraction, or a protein extract. In other embodiments, the sample may be a bodily fluid. Non-limiting examples of suitable bodily fluids include blood, plasma, serum, urine, saliva, sputum, ascites, pleural effusion, or cerebrospinal fluid. The fluid may be used “as is”, the cellular components or exosomoes may be isolated from the fluid, or a protein fraction may be isolated from the fluid using standard techniques.

In some aspects, the biological sample is essentially free of cells. For example, the sample may have less than 10, 9, 8, 7, 6, 5, 4, 3, 2, or 1 cell(s). In one aspect, the biological sample does not contain cells. In certain aspects, the biological sample may be a lymph, saliva, urine, intestinal fluid, or blood (e.g., plasma) sample. In a further aspect, the method may further comprise purifying or isolating an exosome fraction of the sample and/or increasing the production of an exosome fraction of the sample. The exosomal fraction may be isolated from a biological sample by any means known in the art, including, for example with commercially available reagents and kits, or via ultracentrifugation techniques.

As will be appreciated by a skilled artisan, the method of collecting a biological sample can and will vary depending upon the nature of the biological sample and the type of analysis to be performed. Any of a variety of methods generally known in the art may be utilized to collect a biological sample. Generally speaking, the method preferably maintains the integrity of the sample such that incompatible BG-A antigen can be accurately detected and the amount measured according to the disclosure.

Once a sample is obtained, it is processed in vitro to detect and measure the amount of the incompatible BG-A antigen using an anti-incompatible BG-A antibody as described herein. All suitable methods for detecting and measuring an amount of biomarker using an antibody known to one of skill in the art are contemplated within the scope of the invention. Methods for detecting and measuring an amount of biomarker using an antibody (i.e. “antibody-based methods”) are well known in the art. Non-limiting examples include an ELISA, a sandwich immunoassay, an ExoELISA assay, a radioimmunoassay, an immunoblot or Western blot, flow cytometry, immunohistochemistry, and an array.

In general, an antibody-based method of detecting and measuring an amount of the incompatible BG-A antigen comprises contacting some of the sample, the exosomal fraction of the sample or all of the sample, comprising incompatible BG-A antigen with an anti-incompatible BG-A antibody under conditions effective to allow for formation of a complex between the antibody and the incompatible BG-A antigen. Typically, the entire sample is not needed, allowing one skilled in the art to repeatedly detect and measure the amount of the compatible BG-A antigen in the sample. The method may occur in solution, or the antibody or the incompatible BG-A antigen comprising the sample may be immobilized on a solid surface. Non-limiting examples of suitable surfaces may include microtitre plates, test tubes, slides, beads, magnetic beads, resins, and other polymers. Attachment to the substrate may occur in a wide variety of ways, as will be appreciated by those in the art. For example, the substrate and the antibody may be derivatized with chemical functional groups for subsequent attachment of the two. For example, the substrate may be derivatized with a chemical functional group including, but not limited to, amino groups, carboxyl groups, oxo groups or thiol groups. Using these functional groups, the antibody may be attached directly using the functional groups or indirectly using linkers. An anti—the incompatible BG-A antibody may also be attached to the substrate non-covalently. For example, a biotinylated anti-the incompatible BG-A antibody may be prepared, which may bind to surfaces covalently coated with streptavidin, resulting in attachment. Alternatively, an antibody may be synthesized on the surface using techniques such as photopolymerization and photolithography.

Contacting the sample with an antibody under effective conditions for a period of time sufficient to allow formation of a complex generally involves adding the anti-incompatible BG-A antibody composition to the sample and incubating the mixture for a period of time long enough for the anti-incompatible BG-A antibody to bind to any antigen present. After this time, the complex will be washed and the complex may be detected and the amount measured by any method well known in the art. Methods of detecting and measuring an amount of an antibody-polypeptide complex are generally based on the detection of a label or marker. The term “label”, as used herein, refers to any substance attached to an antibody, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni²⁺, Flag tags, myc tags, heavy metals, and enzymes (including alkaline phosphatase, peroxidase, glucose oxidase, and luciferase). Methods of detecting and measuring an amount of an antibody-polypeptide complex based on the detection of a label or marker are well known in the art.

In some embodiments, an antibody-based method is an immunoassay. Immunoassays can be run in a number of different formats. Generally speaking, immunoassays can be divided into two categories: competitive immmunoassays and non-competitive immunoassays. In a competitive immunoassay, an unlabeled analyte in a sample competes with labeled analyte to bind an antibody. Unbound analyte is washed away and the bound analyte is measured. In a non-competitive immunoassay, the antibody is labeled, not the analyte. Non-competitive immunoassays may use one antibody (e.g. the capture antibody is labeled) or more than one antibody (e.g. at least one capture antibody which is unlabeled and at least one “capping” or detection antibody which is labeled.) Suitable labels are described above.

In other embodiments, an antibody-based method is an immunoblot or Western blot. In yet other embodiments, an antibody-based method is flow cytometry. In different embodiments, an antibody-based method is immunohistochemistry (IHC). IHC uses an antibody to detect and quantify antigens in intact tissue samples. The tissue samples may be fresh-frozen and/or formalin-fixed, paraffin-embedded (or plastic-embedded) tissue blocks prepared for study by IHC. Methods of preparing tissue block for study by IHC, as well as methods of performing IHC are well known in the art.

In alternative embodiments, an antibody-based method is an array. An array comprises at least one address, wherein at least one address of the array has disposed thereon an the anti-incompatible BG-A antibody. Arrays may comprise from about 1 to about several hundred thousand addresses. Several substrates suitable for the construction of arrays are known in the art, and one skilled in the art will appreciate that other substrates may become available as the art progresses. Suitable substrates are also described above. In some embodiments, the array comprises at least one anti-incompatible BG-A antibody as described herein is attached to the substrate is located at one or more spatially defined addresses of the array. For example, an array may comprise at least one, at least two, at least three, at least four, or at least five anti-incompatible BG-A antibodies, each antibody recognizing the same or different immune epitopes on BG-A molecules, and each antibody may be at one, two, three, four, five, six, seven, eight, nine, ten or more spatially defined addresses.

Any suitable reference value known in the art may be used. For example, a suitable reference value may be the amount of BG-A antigen in a biological sample obtained from a subject or group of subjects of the same species that has no detectable cancer. In another example, a suitable reference value may be the amount of BG-A antigen in biological sample obtained from a subject or group of subjects of the same species that has detectable incompatible BG-A antigen expressing cancer cell as measured via standard methods such as culture. In another example, a suitable reference value may be a measurement of the amount of incompatible BG-A antigen in a reference sample obtained from the same subject. The reference sample comprises the same type of biological fluid as the test sample, and may or may not be obtained from the subject when the incompatible BG-A antigen expressing cancer was not suspected. A skilled artisan will appreciate that it is not always possible or desirable to obtain a reference sample from a subject when the subject is otherwise healthy. For example, in an acute setting, a reference sample may be the first sample obtained from the subject at presentation. In another example, when monitoring the effectiveness of a therapy, a reference sample may be a sample obtained from a subject before therapy began.

According to the disclosure, a subject may be classified based on the amount of incompatible BG-A antigen measured in the sample. Classifying a subject based on the amount of BG-A antigen measured in a sample of biological fluid obtained from the subject may be used to identify subjects with an incompatible BG-A antigen expressing cancer cells. Generally speaking, a subject may be classified as having a high or low amount of incompatible BG-A antigen compared to a reference value, wherein a high amount of incompatible BG-A antigen is an amount above the reference value and a low amount is an amount equal to or below the reference value. In preferred embodiments, to classify a subject as having a high amount of incompatible BG-A antigen, the amount of incompatible BG-A antigen in the sample compared to the reference value may be at least 5% greater. For example, the amount of incompatible BG-A antigen in the sample may be at least 5%, at least 10%, at least 20%, at least 30%, at least 40%, at least 50%, at least 60%, at least 70%, at least 80%, at least 90%, or at least 100% greater than the reference value. In other embodiments, the amount of incompatible BG-A antigen in the sample of biological fluid obtained from the subject compared to the reference value may be increased at least 2-fold. For example, the amount of incompatible BG-A antigen in the sample compared to the reference value may be increased at least 2-fold, at least 5-fold, at least 10-fold, at least 15-fold, at least 20-fold, at least 25-fold, at least 30-fold, at least 35-fold, at least 40-fold, at least 45-fold, or at least 50-fold.

In another aspect, the invention provides means to detect an incompatible BG-A antigen expressing cancer cell in a subject.

Upon detection of an incompatible BG-A antigen expressing cancer cell, the subject may be treated via methods standard in the art for cancer or the subject may be treated with compositions disclosed herein or a combination thereof. Such treatment methods may depend on the type and severity of an incompatible BG-A antigen expressing cancer cell, as well as the general condition of the patient. Standard treatment of cancer consists primarily of chemotherapeutics, radiation and surgery.

In an embodiment, a method for monitoring an incompatible BG-A antigen levels in biofluid in a subject may be used to determine disease progression or recurrence. In such an embodiment, a method of detecting incompatible BG-A antigen may be used to assess the risk of a subject at one point in time, then at a later time, the method of detecting the incompatible BG-A antigen may be used to determine the change in risk of the subject over time. For example, the method of detecting the incompatible BG-A antigen may be used on the same subject days, weeks, months or years following the initial determination of the amount of the incompatible BG-A antigen. Accordingly, the method of detecting the incompatible BG-A antigen may be used to follow a subject to determine when the risk of progressing to more severe stage of disease is high thereby requiring treatment. Additionally, the method of detecting the incompatible BG-A antigen may be used to measure the rate of disease progression. For example, a depressed amount of the incompatible BG-A antigen may indicate an remission of the disease. Alternatively, an elevated amount of the incompatible BG-A antigen may indicate disease progression. Levels may be monitored, daily, weekly, monthly, yearly, etc. so as to track the progression/remission of an incompatible BG-A antigen expressing cancer cell such as during the period of hospitalization, the duration of treatment, and/or the duration of surviving.

In another embodiment, a method for monitoring the incompatible BG-A antigen levels in biofluid in a subject may also be used to determine the response to treatment. As used herein, subjects who respond to treatment are said to have benefited from treatment. For example, a method to detect the incompatible BG-A antigen may be performed on the biological sample of the subject prior to initiation of treatment, then at a later time, a method to detect the incompatible BG-A antigen may be used to determine the response to treatment over time. For example, a method to detect the incompatible BG-A antigen may be performed on the biological sample of the same subject days, weeks, months or years following initiation of treatment. Accordingly, a method to detect the BG-A antigen may be used to follow a subject receiving treatment to determine if the subject is responding to treatment. If the amount of the BG-A antigen remains the same levels (amount) or decreases, then the subject may be responding to treatment. If the amount of the incompatible BG-A antigen increases, then the subject may not be responding to treatment. These steps may be repeated to determine the response to therapy over time.

For each aspect, the method generally comprises (a) measuring the amount of the incompatible BG-A antigen in a biological sample obtained from a subject using the anti-incompatible BG-A antibody as described herein, and (b) comparing the amount of incompatible BG-A antigen in the sample to a reference value. A greater amount of the incompatible BG-A antigen in the sample compared to the reference value indicates the presence of the BG-A antigen expressing cancer cells. The amount of the incompatible BG-A antigen may be a qualitative, a semi-quantitative or quantitative measurement. Suitable anti-the incompatible BG-A antibodies are described above, as are methods for measuring the amount of the BG-A antigen in a biological sample. In a preferred embodiment, the biological sample is biological fluid selected from the group consisting of urine, sputum, saliva, ascites, pleural effusion, blood, plasma, and serum. In a more preferred embodiment, the amount of the incompatible BG-A antigen is measured in the exosomal fraction of a biological fluid of a subject with type B or type O blood. (The exosome form of the incompatible BG-A is measured in a subject with blood type B or O.)

### (b) Methods of Using CAR T-Cells

In another aspect, the present disclosure provides a method of killing a incompatible BG-A antigen expressing cancer cell, the method comprising contacting the cancer cell with an effective amount of a T cell comprising a chimeric antigen receptor (CAR-T cell), wherein the chimeric antigen receptor specifically binds the incompatible BG-A antigen expressed on a cancer cell. Suitable CAR-T cells are described in detail in Section I.

Contacting a cancer cell with an effective amount of a CAR-T cell generally involves admixing the CAR-T cell and the cancer cell for a period of time sufficient to allow the chimeric antigen receptor of the CAR-T cell to bind its cognate antigen on the surface of the malignant cell. This may occur in vitro or ex vivo. The term “effective amount”, as used herein, means an amount that leads to measurable effect, e.g., antigen-dependent cell proliferation, cytokine secretion, cytotoxic killing, etc. The effective amount may be determined by using the methods known in the art and/or described in further detail in the examples.

In another aspect, the present disclosure provides a method for treating a subject having a incompatible BG-A antigen expressing cancer cell. In some embodiments, the cancer cell is a cancer stem cell. In some embodiments, the cancer cell is a colorectal cancer cell or an ovarian cancer cell. The method comprises administering to the subject a therapeutically effective amount of plurality of chimeric antigen receptor T (CAR-T) cells, each CAR-T cell comprising the same chimeric antigen receptor, wherein the chimeric antigen receptor specifically binds an incompatible BG-A antigen expressed on a cancer cell. Suitable subjects include any mammal, preferably a human, more preferably a human with type B or type O blood. Suitable CAR-T cells are described in detail in Section I. The method may comprise allogenic CAR-T cell therapy or autologous CAR-T cell therapy, though allogenic CAR-T cell therapy may be preferred for the reasons discussed in Section I. The CAR-T cell therapy may be accompanied by other therapies, including but not limited to immunotherapy, chemotherapy or radiation therapy.

The CAR-T cells may be administered in effective doses. The effective dose may be either one or multiple doses, and are sufficient to produce the desired therapeutic effect. A typical dose of CAR-T cells may range from about 1×10⁵-5×10⁷ cells/Kg body weight of subject receiving therapy. The effective dose may be calculated based on the stage of the malignancy, the health of the subject, and the type of malignancy. In the situation where multiple doses are administered, that dose and the interval between the doses may be determined based on the subject's response to therapy.

### III. Kits

In an embodiment, an antibody of the disclosure may be used in a kit to diagnose a incompatible BG-A antigen expressing cancer cell. The BG-A antigen expressing cancer cell may be a breast cancer, lung cancer, head and neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer, leukemia, myeloma or skin cancer. In certain aspects, the cancer is a colorectal cancer. Such kits are generally known in the art and commonly used to detect an antigen of interest. These diagnostic kits will generally include the antibodies of the disclosure along with suitable means for detecting binding by that antibody such as would be readily understood by one skilled in this art. For example, the means for detecting binding of the antibody may comprise a detectable label that is linked to said antibody. Non-limiting examples of suitable labels include enzymes, radioactive isotopes, fluorescent compounds, chemical compounds, and bioluminescent proteins. In addition, these kits may include reagents useful in the purification and/or isolation of exosomes. These kits can then be used in diagnostic methods to detect the presence of an incompatible BG-A antigen expressing cancer cell wherein a sample is collected from a subject suspected of having cancer.

Additionally, kits may include the bispecific anti-incompatible BG-A antibodies as described herein or the CAR T-cells as described herein in addition to reagents and a means of administering the compositions.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

These examples provide for the generation of monoclonal antibody (mAb) to incompatible BG-A antigen and evaluate the ability of mAb to detect cancerous and pre-cancerous cells in vitro and in vivo. Briefly, mAb CRC-A1 was successfully generated. Analysis of tumor and normal tissues, including peripheral blood mononuclear cells, showed that mAb CRC-A1 had excellent tumor sensitivity, homogeneity, and significant tumor specificity. The ability of mAb CRC-A1 to target both cancer stem cells (CSCs) and differentiated tumor cells provides an exciting opportunity to image and treat the whole tumor cell population in a clinical setting. Moreover, compositions comprising the mAb CRC-A1 or fragments thereof development immunotherapeutic reagents for cancer treatment will potentially improve the quality of life and increase the survival rate for cancer patients.

### Example 1—Binding of mAb CRC-A1 to the Membrane of Human Colon Adenocarcinoma NSY Cells and Human Colon Cancer Tissue Array Analysis

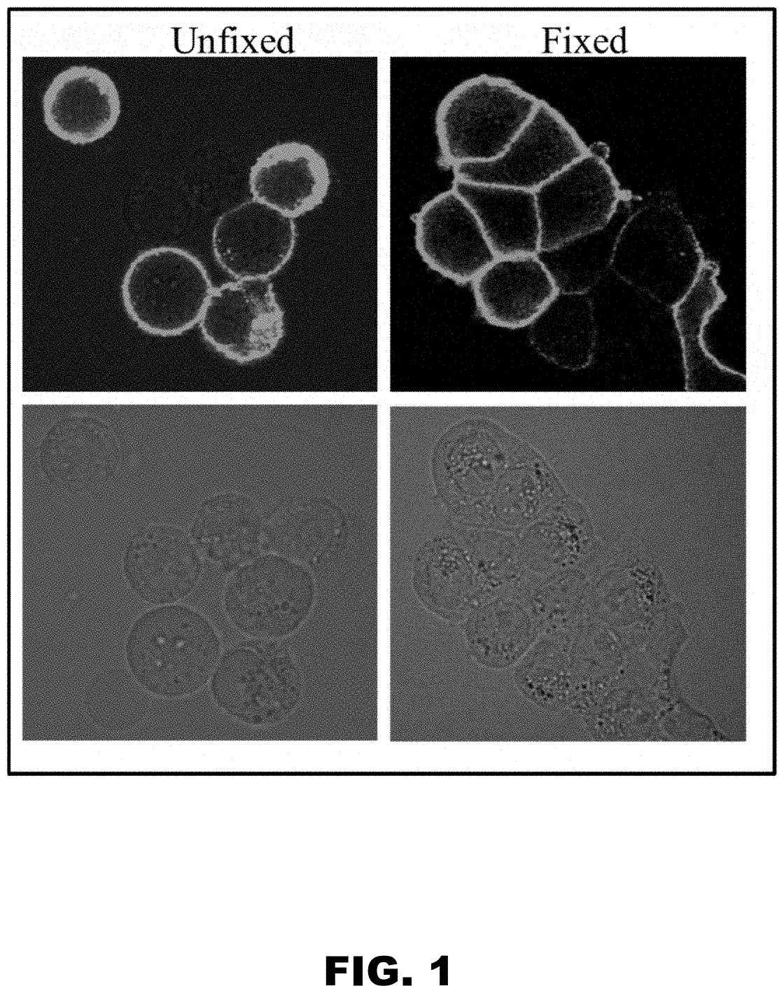

To examine if mAb CRC-A1 binds antigens expressed on cell surface, immunofluorescence staining in cultured cells without fixation and permeation was performed. Very intense cell membrane staining and internalization of mAb CRC-A1 were observed in >80% of the cultured live NSY cells after incubation at 37° C. for 40 min (FIG. 1, top left). To further examine the cellular localization of the mAb CRC-A1 binding epitope, immunofluorescence staining in cells fixed and permeated with 4% paraformaldehyde containing 0.2% Triton X-100 was performed. mAb CRC-A1 was strongly reactive to the membrane of NSY cells (FIG. 1, top right).

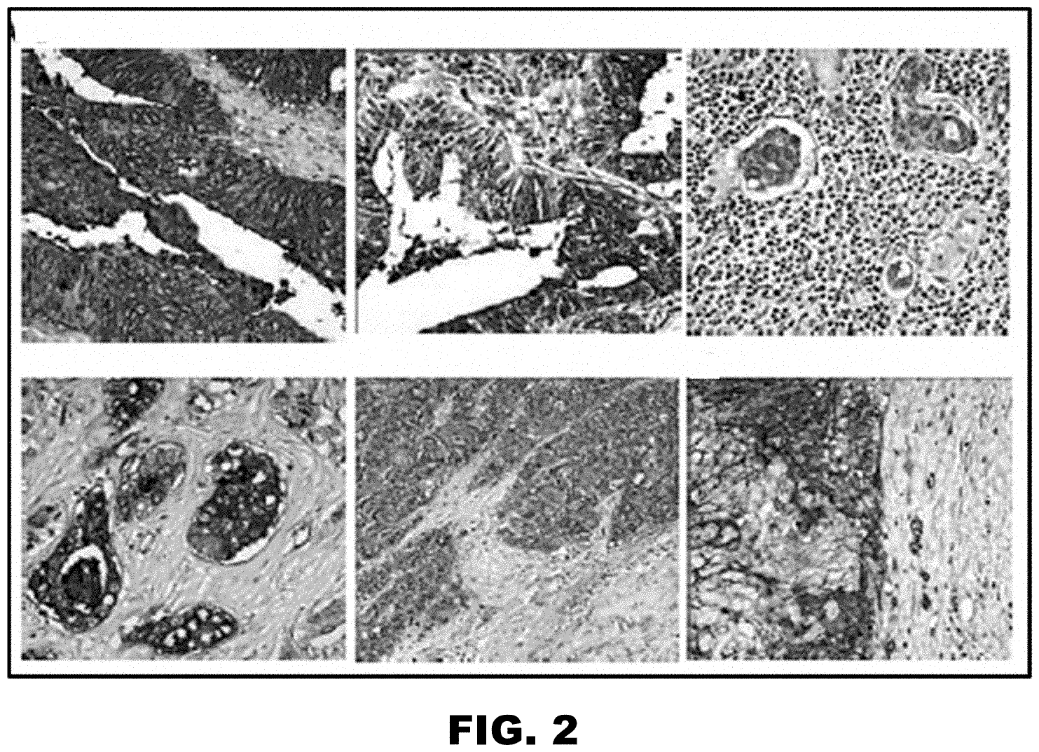

To evaluate the usefulness of an antibody for tumor imaging and targeted therapy, it is necessary to test tumor sensitivity, homogeneity, and specificity of the epitope recognized by the antibody in human tumors. At first, the reactivity of the antibody in human colon cancer tissues was examined with mAb CRC-A1. It was found that 98.9% (187 of 189 cases) of colon cancer showed positive staining with mAb CRC-A1. “+”, “++,” and “+++” were 25.9%, 28.6%, and 43.4%, respectively (Table 1). The results showed that >40% of all colon cancer cases tested exhibited intense homogenous staining, and the epitope recognized by mAb CRC-A1 was expressed in all histologic grades of colorectal cancers, as shown in (FIG. 2). The immunoepitope was also detected in tumor cells that metastasized into lymph nodes (FIG. 2).

TABLE 1
Summary of mAb CRC-A1 Reactivity in
Human Colorectal Tumor Tissues.

| | Scoring Grade | | | | | |
|---|---|---|---|---|---|---|
| | − | ± | + | ++ | +++ | Total (%) |
| Cases | 2 (1.05) | 2 (1.05) | 49 (25.9) | 54 (28.6) | 82 (43.4) | 189 (100) |
| (%) | | | | | | |

### Example 2—mAb CRC-A1 Binds to BG-A and BG-A1 Antigens

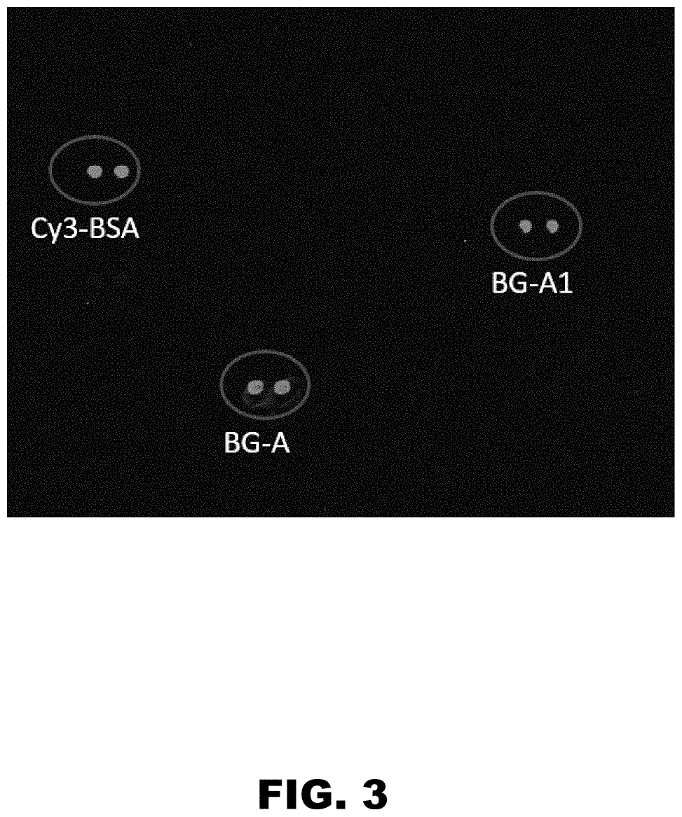

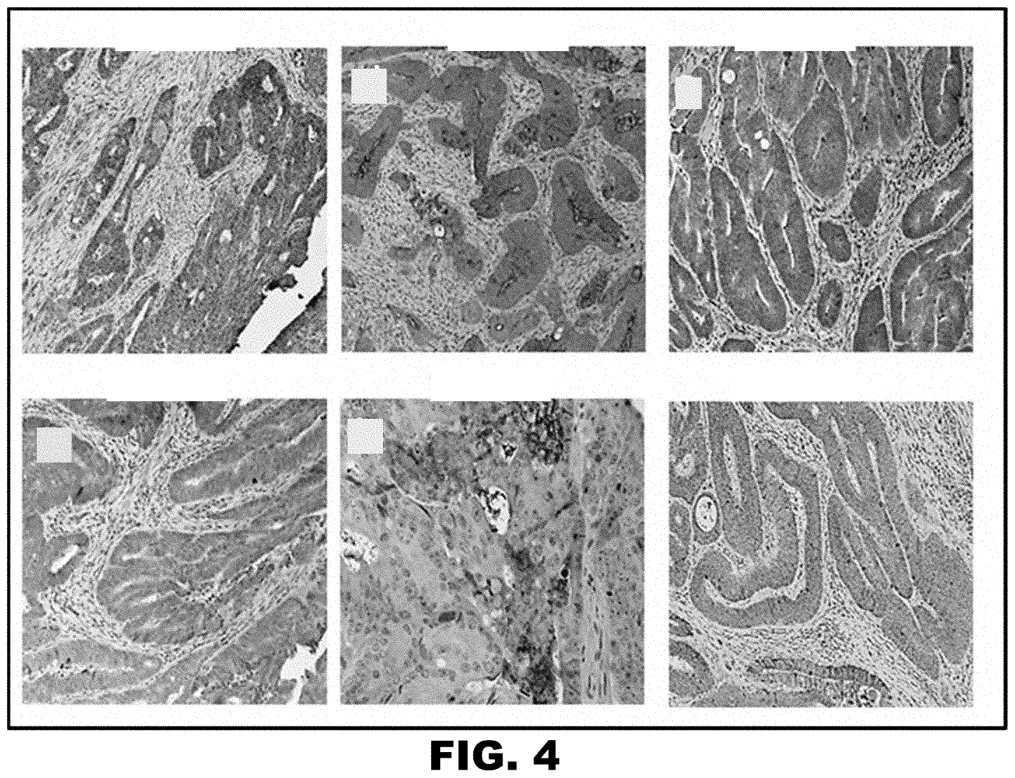

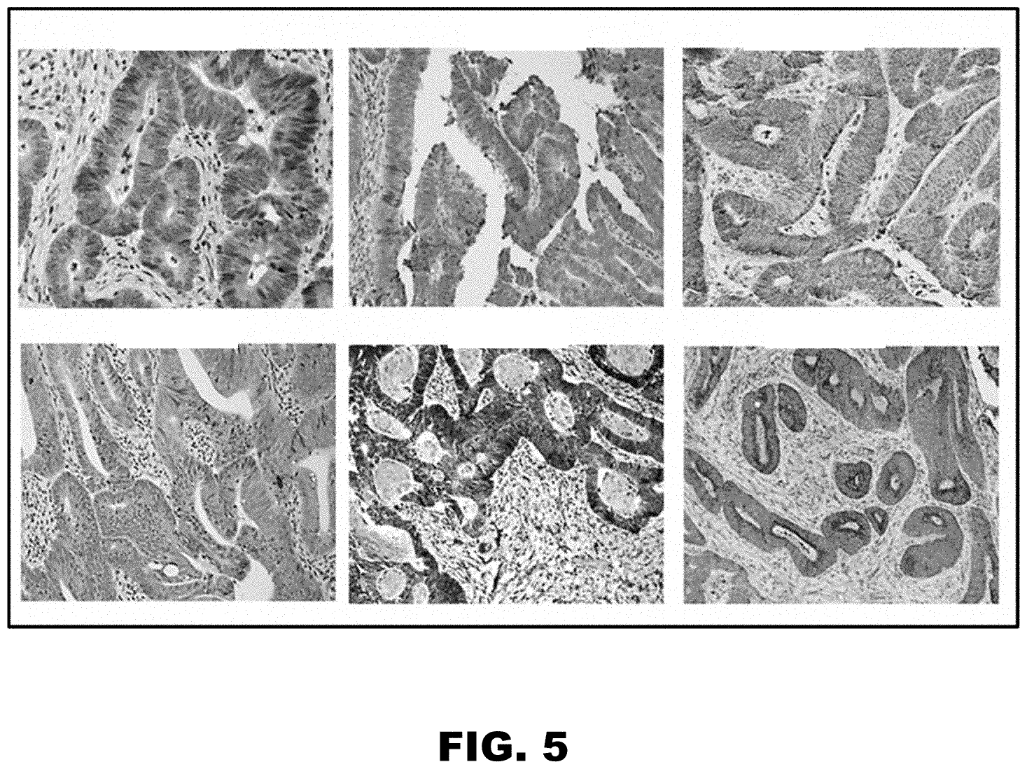

Presently, most of tumor antigens identified are tumor associated antigens (TAAs) those over express on tumor cells and also present on some normal cells as well. In recent years, the concept of precision (personalized) medicine has been proposed as a promising approach for treatment of cancer. Some TAAs may not tumor specific in general colorectal cancer (CRC) populations, but they become tumor specific in a certain sub-group of CRC. To identify and characterize the binding epitope of CRC-A1 an antigen array of about 200 different glycans and tumor associated antigens was performed. As can be seen in FIG. 3, CRC-A1 binds to BG-A and BG-A1. Interestingly, these results provided the opportunity to detect specific tumor antigen in individuals with blood type O and B. To test this concept immunohistochemical staining with CRC-A1 of colorectal cancer tissues from patients with blood type B (FIG. 4) and blood type O (FIG. 5) was performed. As can be seen in FIG. 4 and FIG. 5, a novel binding epitope on the incompatible BG-A molecules with 100% (16/16) and 92% (23/25) positive rates for colorectal cancer patients with BG-B and BG-O phenotypes respectively. Indeed, healthy individuals with blood type O and B have blood group H (BG-H) and blood group B (BG-B) antigen, but absolutely have no BG-A antigen in their cells and tissues. However, their CRC cells can produce and express BG-A antigen that is called incompatible BG-A antigen that is absolute tumor specific for CRC patient with blood type B and 0.

### Example 3—Expression of the Immune Epitope Recognized by mAb CRC-A1 within the Incompatible BG-A on the Surface of Pre-Cancerous Adenomas and CSCs

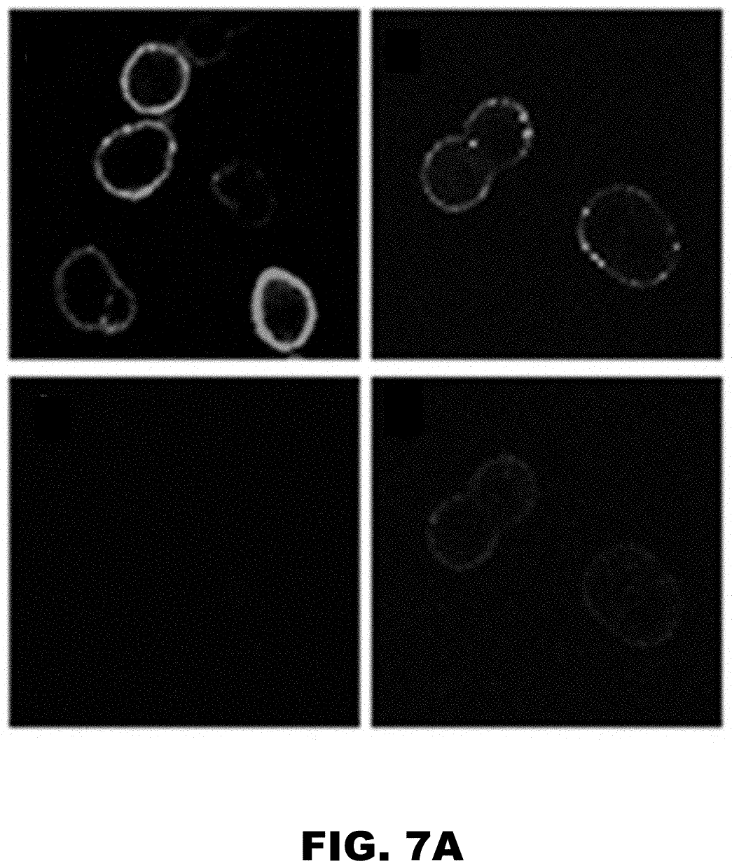

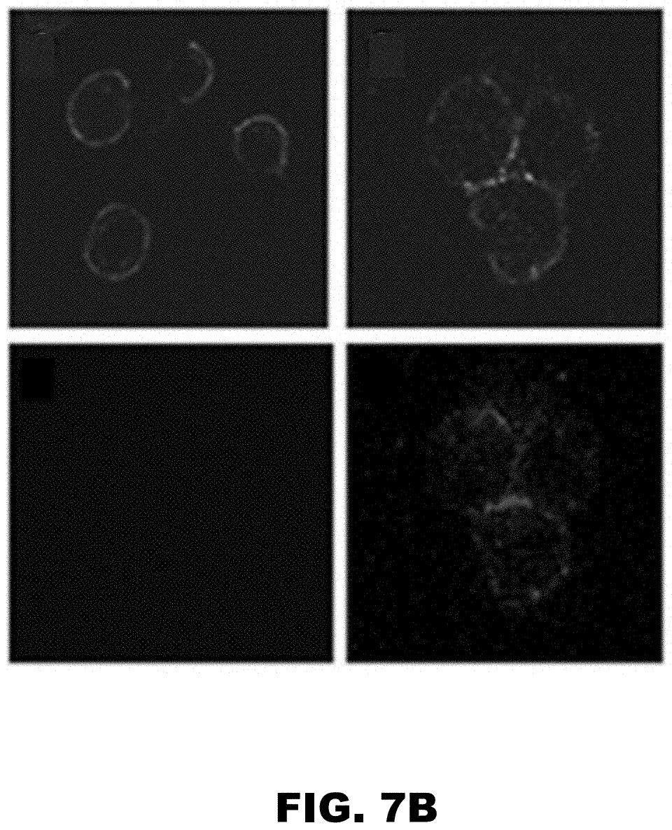

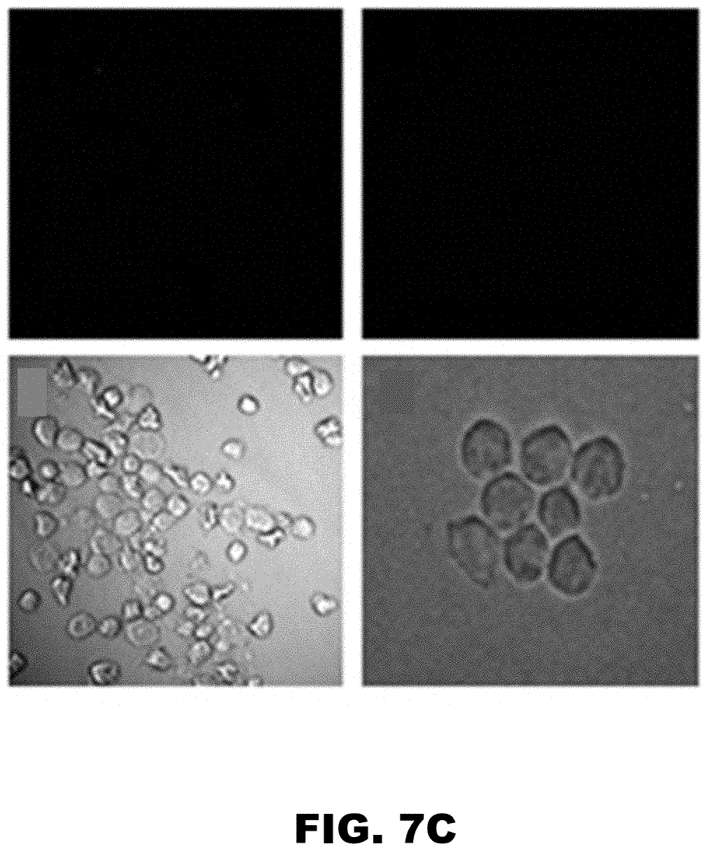

Considering that CSCs are the source of the whole population of tumor, they constitute an important target for tumor imaging and therapy. Because mAb CRC-A1 showed intense staining in colon cancer tissues, we investigated the expression of the immune epitope in colon CSCs. The colon CSCs were isolated based on CD133 expression (antibody to CD133/1binding site) and stained with mAb CRC-A1 labeled with Alexa Fluor 488 and phycoerythrinconjugated mAb 293C3 against CD133/2 binding site to confirm the identity of the CSCs. Impressively, mAb CRC-A1 not only stained the surface of differentiated (CD133−) colon cancer cells, but also strongly stained the membrane of colon cancer stem (CD133+) cells (FIG. 7). For tumor molecular imaging and targeted therapy, it is important that the epitope recognized by mAb CRC-A1 is not expressed in peripheral (circulating) blood mononuclear cells. The result show that the peripheral blood mononuclear cells were negatively stained (FIG. 7).

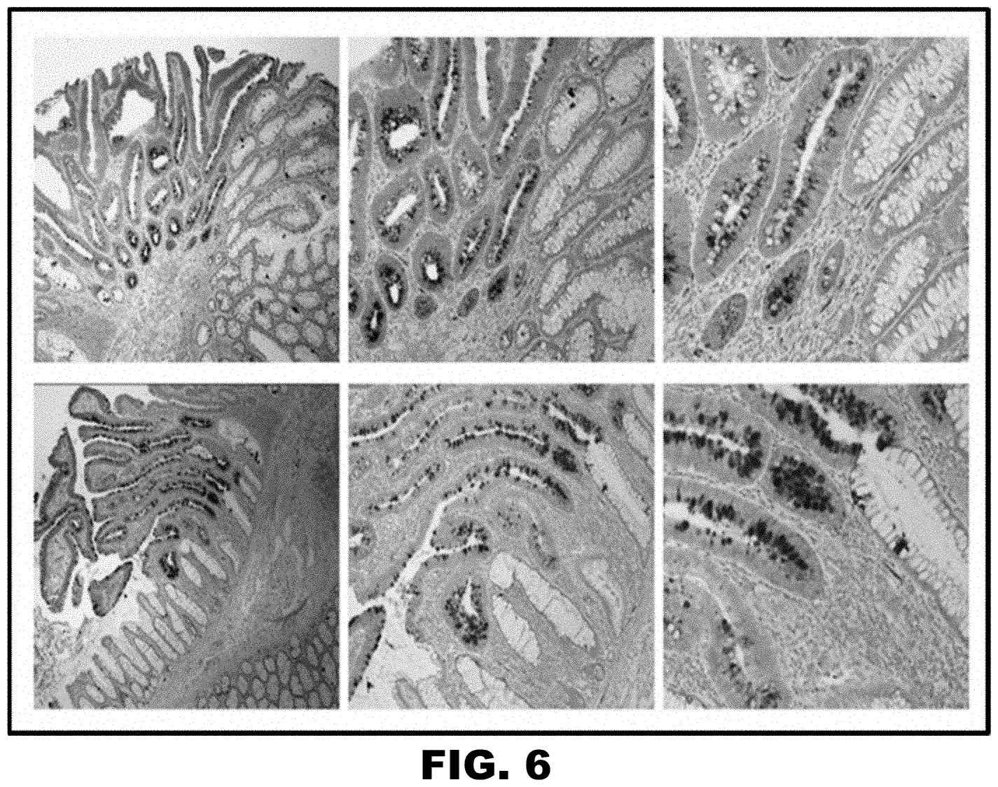

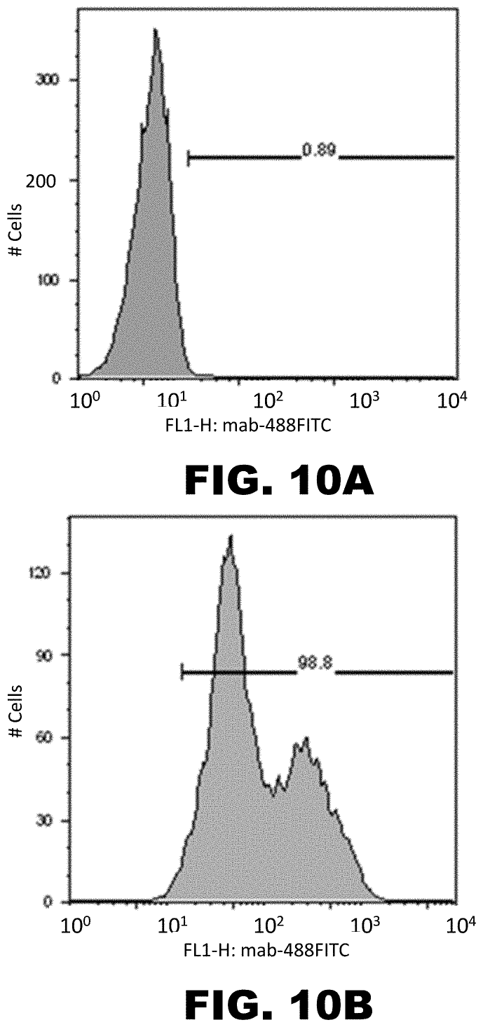

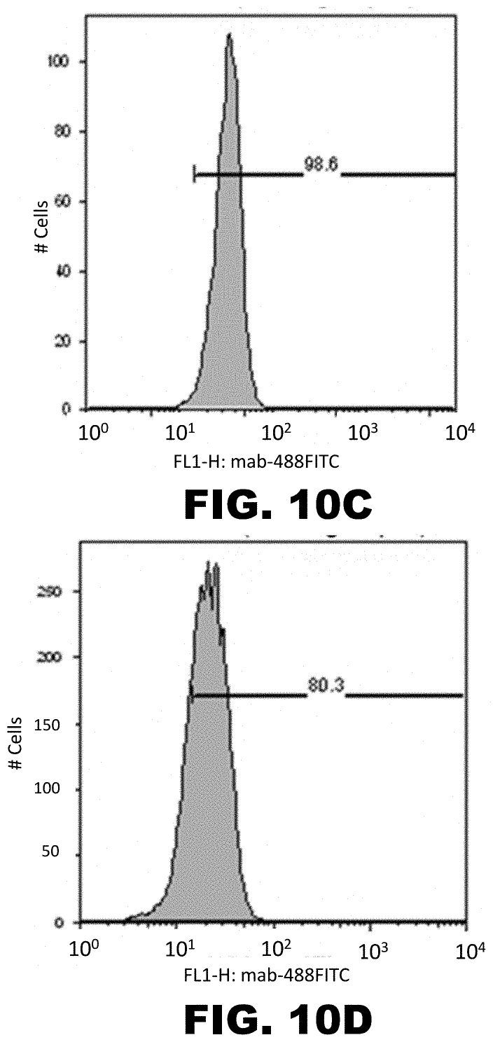

Moreover, CRC-A1 staining of pre-cancerous tissues from patients with blood type B or O showed positive staining providing further support in the ability of CRC-A1 to detect cancer cells and pre-cancerous cells (FIG. 6). Lastly, flowcytometry analysis of Mab CRC-A1 show that Mab CRC-A1 react to AML cells from patients with Blood type B, indicating that CRC-A1 utility is not limited to colon and ovarian cancer but instead can be utilized for any cancer or precancer which expresses or secrets an incompatible BG-A antigen (FIG. 10).

### Example 4—the Binding Epitope of mAb CRC-A1 is Different from Those of Anti-BG-A Antibodies Naturally Contained in Blood Type B and O Serum

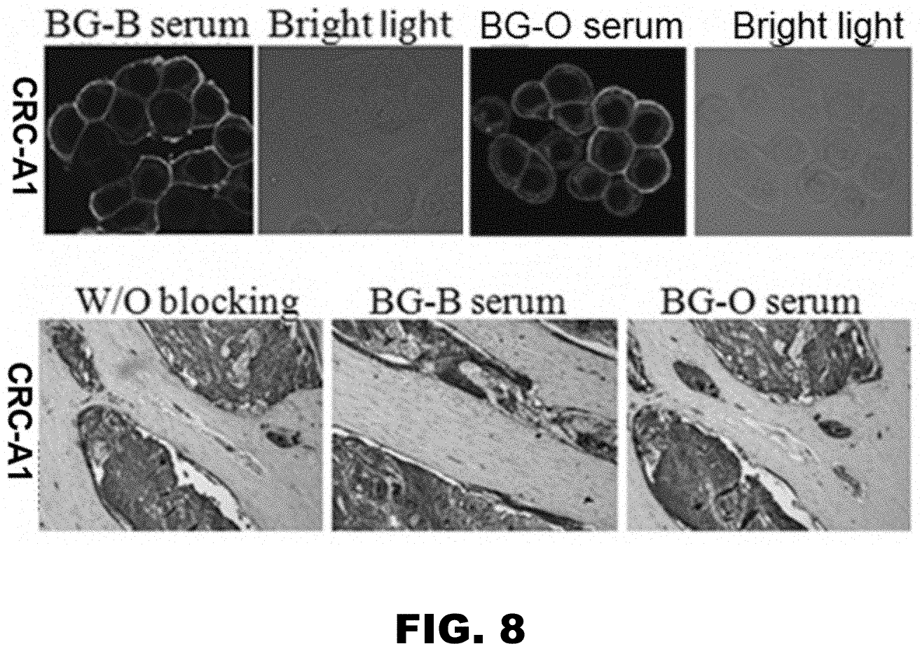

mAb CRC188 positively stained colon cancer stem (CD133+) cells (FIG. 6) and the staining could not be blocked by blood type B and O serum that contains antibodies to BG-A antigen. The results indicate that the binding site or immune epitope of mAb CRC-A1 is different from those reacted to BG-A antibodies naturally contained in blood type B and O serum. In other words, the mAb CRC-A1 binding epitope carried by BG-A molecules are not exposed to immune system in normal condition (FIG. 8).

### Example 5—Biodistribution and Kinetic Changes of Mab CRC-A1 Labeled with Near Infrared (NIR) Dye Imaging Probes In Vivo

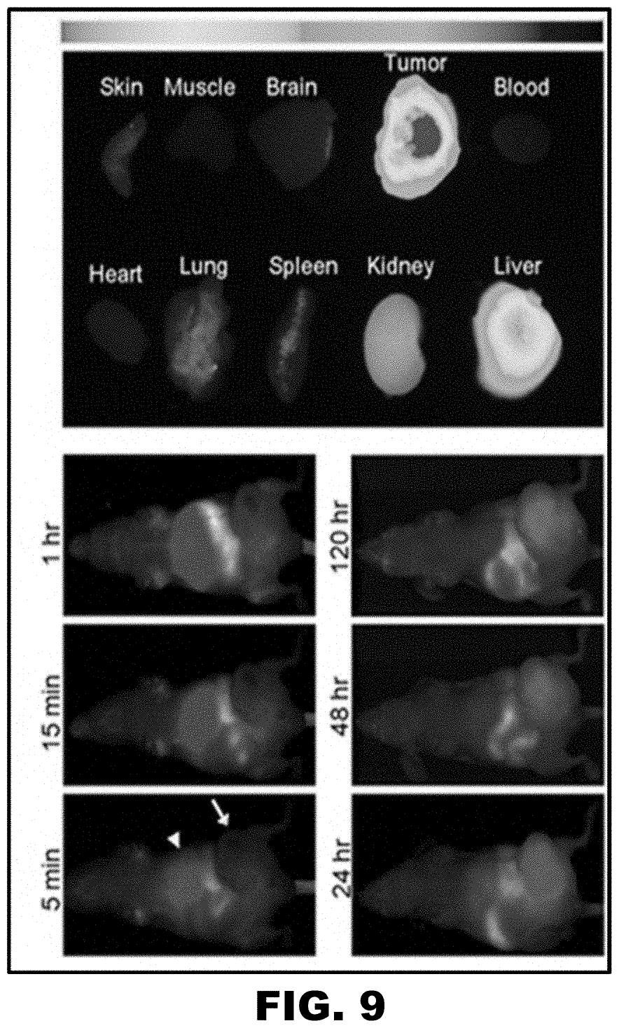

A major motivation for identifying tumor specific binding ligands including antibodies is for the diagnosis and treatment of cancer. Evaluation of imaging and therapeutic reagents in the experimental animal is a critical stage for developing biological reagents for future clinical use. Therefore, Mab CRC-A1 was labeled with a NIR dye IRDye 800CW and the biodistribution and kinetic changes of the antibody-NIR dye conjugates in colon tumor bearing mice was monitored. As demonstrated in FIG. 9, Left panel: Significant amounts of the conjugates accumulate in excretion organs (liver) at 5 minutes, 15 minutes and 1 hour post injection. At 24 hours, the imaging conjugates predominantly accumulated in the tumor, while the intensity of the imaging probes was decreased in the liver. At 120 hours post injection, a significant amount of the imaging probe was still retained in the tumor. Right panel: Ex vivo images, the Mab CRC-A1 conjugates mainly maintained in tumor tissues at 120 hours post injection. Arrow head: liver; Arrow: tumor.

In this study, we have observed that the probes penetrate throughout the tumor within 24 hours post injection, particularly diffusing into the tumor core areas. Moreover, the probe is retained in the tumor tissue for at least 120 hours. We found that once the imaging probes reach the core areas, they accumulate and are retained there for a longer period of time than when they are bound to peripheral tumor tissues and excretory organs such as the liver and kidney. Mab CRC-A1 based imaging and therapeutic reagents may be capable of detecting subclinical tumors in vivo and efficiently delivering anti-cancer drugs to the tumor tissues. Antibody based imaging and therapeutic reagents have advantages and disadvantages in tumor targeted imaging and therapy. The conjugates of antibody with imaging agents and anti-cancer drugs generally have higher binding affinity and specificity. They are also retained in the tumor for a much longer time compared to small peptide based imaging and therapeutic reagents. The disadvantage is that longer time is required for clearance from normal cells and tissues including blood and excretory organs. There are also some concerns about diffusion of antibody based imaging and therapeutic reagents into deep tumor tissues where there is a higher interstitial fluid pressure and heterogeneous blood supply. However, our results demonstrate that mAb CRC-A1-NIR conjugates penetrated into the tumor tissue core areas within 24 hours.

Furthermore, to shorten the clearance time and reduce the binding of antibody based imaging and therapeutic reagents to normal tissue, it is possible to develop several tumor pre-targeting systems and IgG fragments based imaging and therapeutic reagents such as Fab, F(ab′)2, scFv, diabodies and minibodies.

### Example 6—Developing Immunotherapeutic Reagents for Cancer Treatment

As shown above, a hybridoma that produces mAb CRC-A1 has been created and identified a novel tumor specific immune-epitope carried on BG-A molecules. In addition, compositions comprising Mab CRC-A1 gene for generation of immunotherapeutic agents BiTE and CAR-T cells as well as CDR peptides are envisioned.

Antibody and its fragment based Immunoconjugates are specific, highly effective, minimally toxic anticancer agents that are beginning to show promise in the clinic. Immunoconjugates consist of three separate components: an antibody that binds to a cancer cell antigen with high specificity, an effector molecule that has a high capacity to kill the cancer cell, and a linker that will ensure the effector does not separate from the antibody or its fragments during delivery and will reliably release the effector to the cancer cell or tumor stroma. For example, a conjugate mAb CRC-A1 with monomethyl auristatin E (MMAE) via hydrazone bond linker to create tumor targeted agents for cancer treatment. Hydrazone bond linkers are stable in the neutral pH of the plasma and degraded once internalized into tumor cells (exposed to the acidic pH of lysosomes), releasing the payload (MMAE) that is the therapeutic entity. Note that Brentuximab vedotin, a hydrazone bond linked anti-CD30 antibody with MMAE, has been approved by the FDA for the treatment of certain lymphomas and breast cancers.

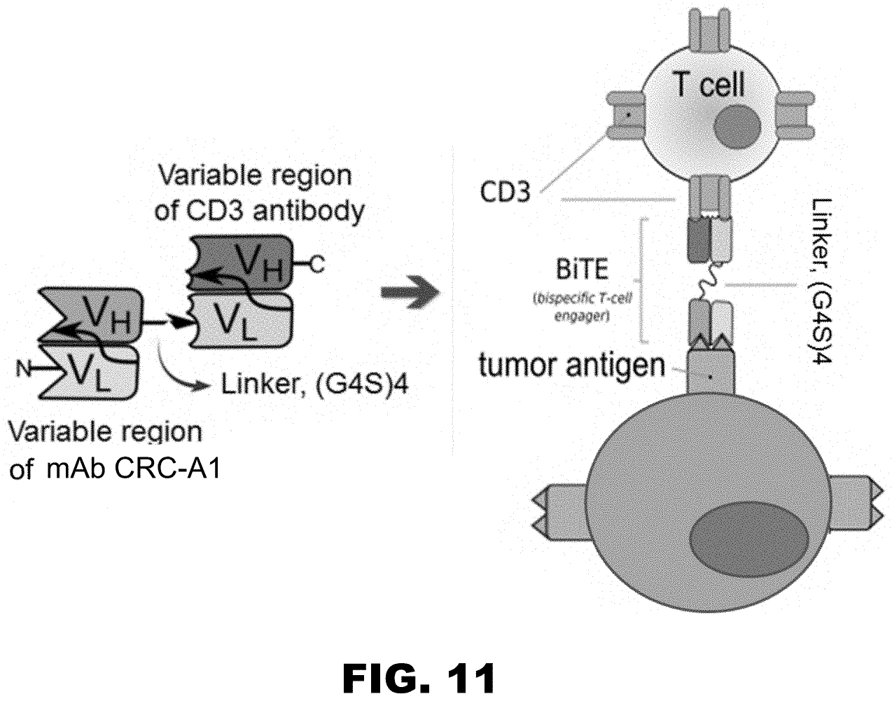

Generation of BiTE: we will choose commonly used genetic engineering method and single-chain-based format (FIG. 11) to generate BiTE. Briefly, molecular cloning technology to clone cDNAs of encoding the variable domains of each parental monoclonal antibody and linkers, and then link them together to form a single-chain bispesific antibody (VLA-linker1-VHA-linker2-VHB-linker3-VLB) is used. VL and VH are derived from the single chain antibody fragment (scFV). A and B represent the parental monoclonal antibody A (CRC-A1) and B (CD3 antibody) (FIG. 11).

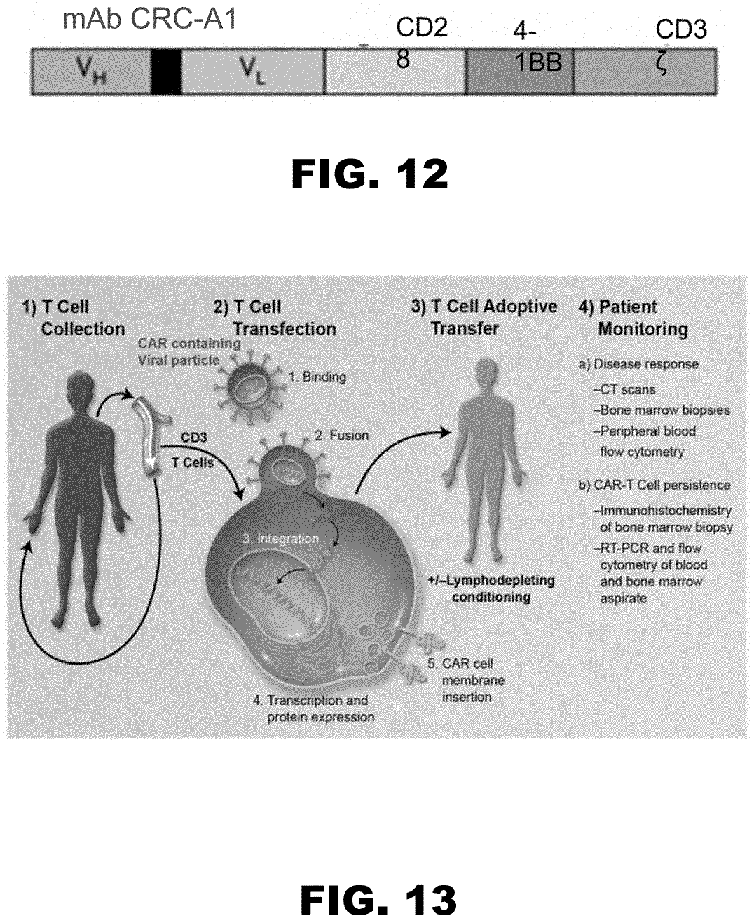

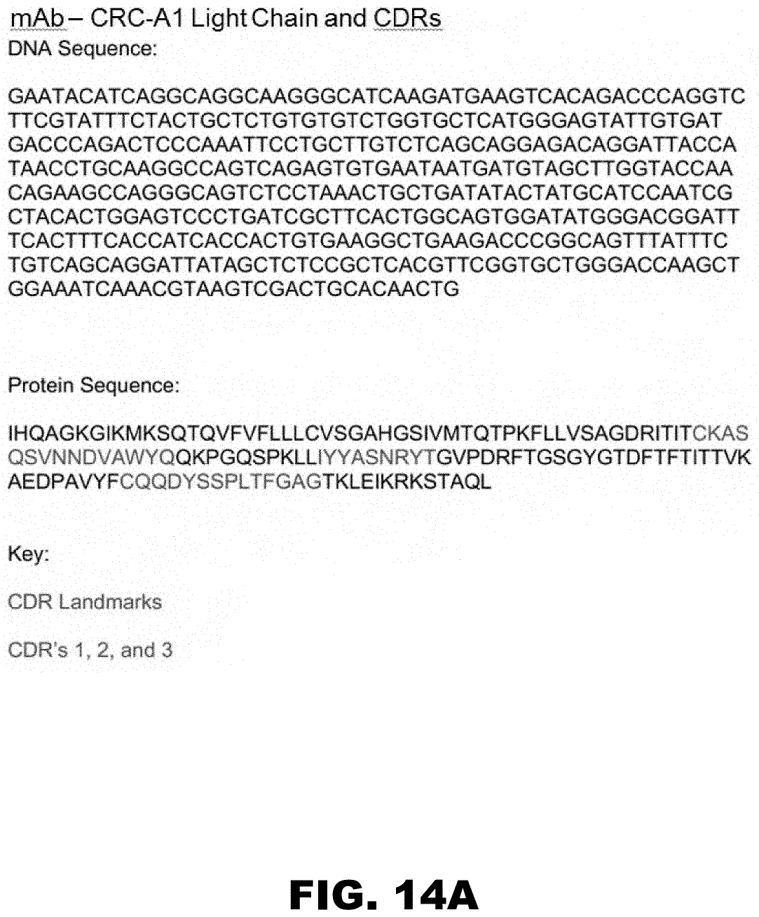

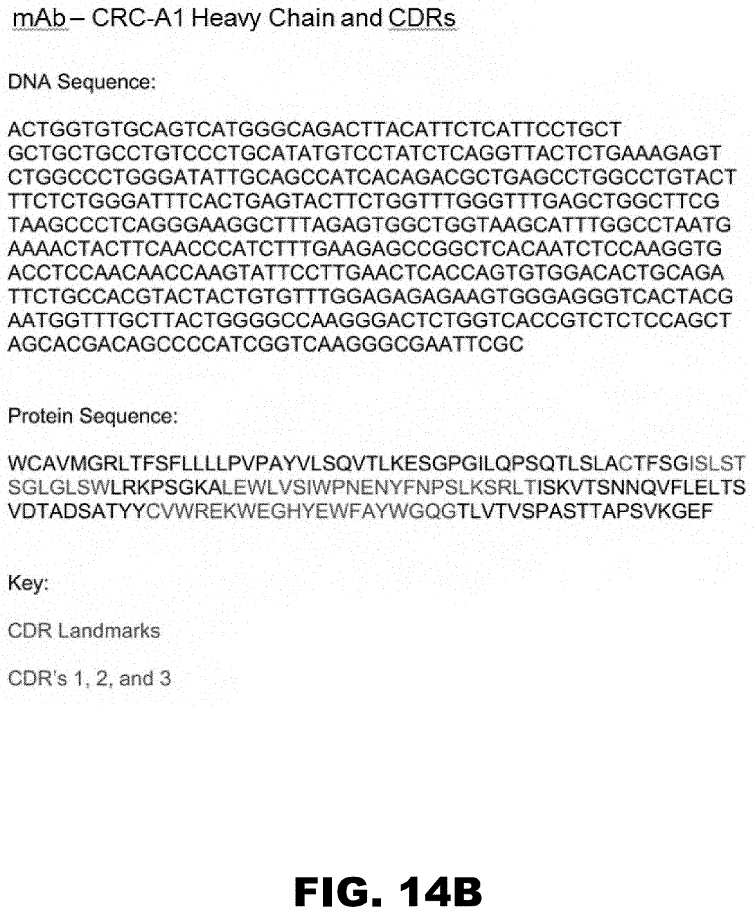

Development of CAR-T cell: As stated above, we have created a hybridoma that produce a monoclonal antibody CRC-A1, identified a tumor cell surface biomarker incompatible BG-A, and sequenced the Mab CRC-A1 gene. To generate a CAR, first construct scFvs of Mab CRC-A1 is generated, and then insert the scFvs gene into pCDCAR9 vector containingCD28-CD3ζ (FIG. 12). For transfer CAR gene into T cells, CAR is packaged with lentiviruses that have been clinically validated. Outlines of CAR-T cell adoptive therapy in cancer patients are shown in FIG. 13.

### Materials and Methods of Examples 1-6

Cell Lines and Cell Culture:

Human colon adenoma cell line NSY was cultured in RPMI 1640 supplemented with 10% FCS (Hyclone), 50 units/mL sodium penicillin, and 50 Ag/mL streptomycin sulfate (BioWhittaker). Myeloma cells (P3/x63.Ag8) were used as a fusion partner and were maintained in Iscove's modified Dulbecco's medium supplemented with 20% fetal bovine serum (Hyclone), 50 units/mL sodium penicillin, 50 Ag/mL streptomycin sulfate, 4 mmol/L L-glutamine (BioWhittaker), 1 mmol/L sodium pyruvate (BioWhittaker), and 0.0001% h-mercaptoethanol (Sigma Chemical Co.). Human ovarian cancer A2780 cells were cultured in RPMI1640 medium supplemented with 10% FCS (Hyclone), 50 units/ml sodium penicillin, 50 pg/ml streptomycin sulfate (BioWhittaker) and 2 mM glutamine. SKOV-3 human ovarian cancer cells were maintained in DMEM medium supplemented with 10% FCS, 50 units/ml sodium penicillin, 50 pg/ml streptomycin sulfate (BioWhittaker). mAb CRC-A1 hybridoma cells were cultured in Iscove's modified Dulbecco's medium supplemented with 20% fetal bovine serum (Hyclone), 50 units/ml sodium penicillin, 50 pg/ml streptomycin sulfate, 4 mmol/l L-glutamine (BioWhittaker), 1 mmol/l sodium pyruvate (BioWhittaker), and 0.0001% h-mercaptoethanol (Sigma Chemical Co.) for the antibody production. All cells were cultured in a humidified incubator at 37° C. with 5% CO₂.

Monoclonal Antibody Production and Purification:

Monoclonal antibody (mAab) CRC-A1 was produced by culturing the hybridoma cells in vitro and purified using protein G Sepharose according to the manufacturer's instruction (Amersham Biosciences). Briefly, the supernatant from the hybridoma culture was centrifuged at 14,000 g for 20 minutes at 4° C., filtered through a 0.22-μm filter to remove fine particles, and the pH was adjusted to 7.0 using equilibration buffer [1 mol/l Tris (pH 9.0)]. The supernatant was passed through a protein G Sepharose column (GE healthcare, Life Sciences) and the column was washed with binding buffer [50 mmol/l Na2PO4, 500 mmol/l NaCl (pH 6)] before eluting the antibody with glycine (0.1 mol/l; pH 2.7). The antibody was collected and neutralized in a test tube containing 100 μl of equilibration buffer (1 mol/l, pH 9.0). Antibody concentration was determined using a UV/vis spectrophotometer (Beckman DU 640).

Immunofluorescence Staining Assay, Antibody Labeling, and Microscopy:

For screening hybridoma, we applied immunofluorescence staining assay in cultured cells without fixation and permeation. Under this staining condition, only cell surface molecules can be detected. Briefly, we seeded 5,000 cells per well in 96-well plates and allowed them to continuously culture in an incubator at 37° C. for 48 h. After decanting the culture medium, 50 μL supernatants from the hybridoma culture were added and incubated for 40 min at 37° C. The plates were washed thrice with PBS, and 50 μL of 1,000 diluted Alexa Flour 488-conjugated goat anti-mouse IgG antibody (Molecular Probes) were added to the plates, which was incubated for another 40 min at room temperature. The plates were washed with PBS and observed under a fluorescence microscope. To examine the reactivity of mAb CRC-A1 to colon CSCs, the direct immunofluorescence method was used. mAb CRC-A1 was labeled with Alexa Fluor 488 following the manufacturer's instruction (Invitrogen). Briefly, bicarbonate (50 μL; pH 9.0; 1 mol/L) was added to mAb CRC-A1 (0.5 mL; 2 mg/mL) to optimize the pH for efficient reaction of Alexa Fluor 488 dye and the protein. The antibody solution was transferred to a vial containing the reactive dye and the mixture was incubated for 1 h at room temperature before passing it through a purification column from the labeling kit. The first fluorescence band was collected for immunofluorescence staining. For confocal microscopy, cells cultured on Lab-Tek slides or sedimented by using Cytospin (Thermo Shandon, Inc.) were visualized with an Olympus FV1000 microscope. Dual color, Alexa Flour 488-conjugated mAb CRC-A1 and phycoerythrin-conjugated mAb 294C3 (Miltenyi) staining slides were analyzed using a sequential program from Olympus FV1000 microscope software.

Immunohistochemical Staining

Normal and tumor tissue arrays (Biomax), as well as histologic sections of colon cancer tissue, were used in this study. Histopathologic slides were deparaffinized in xylene and gradient alcohol. After rehydration, the slides were immersed in antigen retrieval solution [10 mmol/L sodium citrate, 0.05% Tween 20 (pH 6.0)] preheated to 95° C. to 100° C. for 20 min. The buffer and the slides were cooled to room temperature and the slides were rinsed twice with PBS before incubation in blocking buffer containing 5% goat serum for 30 min. The slides were further incubated with a primary antibody at a concentration of 10 pg/mL for 1 h at room temperature or overnight at 4° C. After rinsing the slides and blocking with peroxidase blocking solution (3% H₂O₂ in PBS) for 10 min, the Avidin-Biotin Complex detection system (Vector Laboratory) was used following the manufacturer's instruction. 3,30-Diaminobenzidine tetrahydrochloride dehydrate (Sigma Chemical Co.) was used as chromogen. The stained slides were dehydrated with gradient alcohol and xylene. Finally, the slides were sealed with mounting medium, coverslipped, and scored independently by two researchers with pathology background using a conventional microscope. Based on the staining intensity, all cases were scored as negative (grade 0), weak or faint (grade 1), positive (grade 2), strong positive (grade 3), and very strong positive (grade 4) staining. Based on the cell staining proportion, all cases were classified as no staining (−), <5% (F), 6% to 25% (+), 26% to 70% (++), and >71% (+++). A combination of both staining intensity and percentage resulted in the following classifications: grade 0 or no staining (−); grade 1 or (<5%); and the rest of the cases with positive (grade 2), strong positive (grade 3), and very strong positive staining (grade 4) were classified according to the percentage of positively stained cells.

Labeling of Mab CRC-a1 (IgG with NIR Fluorescent Dye for Imaging In Vivo:

For tumor optical imaging in a tumor bearing mouse, we labeled mAb CRC-A1 with NIR dye using an IRDye 800CW Protein Labeling Kit (LI-COR Biosciences, Lincoln, Nebr.) following manufacturer's instructions. Briefly, IRDye dye in a tube was dissolved in 25 μl of ultra pure water provided in the kit and mixed thoroughly by vortexing. Dye solution (7.2 μl) from the tube reacted with 1 ml mAb CRC-A1 solution (1 mg/ml, free of ammonium ions, primary amines and preservatives such as sodium azide by dialysis and gel filtration). The mixture was incubated for 2 hours at 20° C. with protection from light. The dye-antibody conjugate was isolated from free dye using the Pierce Zeba Desalting Spin Column that came with the labeling kit. To verify the labeling quality, we measured the absorbance of the solution containing conjugates at 280 nm and 780 nm (A280 and A780) and calculated the moles of dye per mole of protein according to the formula provided by LI-COR Biosciences. The labeling ratio of dye/protein is 1-2/1.

Optical Imaging of Tumor In Vivo:

BALB/c background nude mice were used in this study (n=2). 1×10⁷ NSY human colon cancer cells were injected subcutaneously at right flank. For non-invasive optical imaging, 100 pg of dye-labeled mAb CRC-A1 in 100 μl of PBS solution was administered per tumor bearing animal by tail vein injection. The animal was scanned with the Pearl NIR imaging system (LI-COR Biosciences, Lincoln, Nebr.) at indicated time points. At the end of experiment (120 hours post probe injection), ex-vivo biodistribution of the imaging reagents was assessed to confirm the noninvasive in vivo observation. Briefly, mice were euthanized by cervical dislocation under isoflurane anesthesia at 120 hours post injection of the imaging reagent. Aliquots of blood and portions of major organs (heart, kidney, lung, spleen, brain. etc) were harvested, washed with PBS and dabbed dry. The Pearl Imager was used to uniformly excite the tissues and organs at the appropriate wavelengths.

1. A method of detecting an immune epitope or immune-determinant carried by an incompatible blood group A (BG-A) antigen in a subject comprising the steps; (a) obtaining a biological sample from a subject, (b) isolating an exosomal fraction from the biological sample and (c) determining the presence or absence of the incompatible BG-A antigen with an anti-incompatible BG-A antibody.

2. The method of claim 1, further comprising the step (d) identifying or diagnosing a subject as having an incompatible BG-A antigen expressing cancer cell when the antibody recognizes an immune epitope carried by incompatible BG-A antigen present in the biological sample.

3. The method of claim 2, wherein the subject has blood type B or type O phenotype.

4. The method of claim 1, wherein the biological sample is a biological fluid.

5. The method of claim 2, wherein the subject has or is suspected of have a cancer cell, wherein the cancer cell is a skin cancer cell, breast cancer cell, lung cancer cell, head and neck cancer cell, prostate cancer cell, esophageal cancer cell, tracheal cancer cell, brain cancer cell, liver cancer cell, bladder cancer cell, stomach cancer cell, pancreatic cancer cell, ovarian cancer cell, uterine cancer cell, cervical cancer cell, testicular cancer cell, colon cancer cell, rectal cancer cell, blood cancer cell such as leukemia and multiple myeloma.

6. The method of claim 5, wherein the cancer cell is a colorectal cancer cell.

7. The method of claim 1, wherein the anti-incompatible BG-A antibody is CRC-A1.

8. The method of claim 1, wherein the anti-incompatible BG-A antibody comprises one or more CDRs from the heavy chain variable region comprising SEQ ID NO:4 and/or one or more CDRs from the light chain variable region comprising SEQ ID NO:2.

9.-27. (canceled)

28. A T cell comprising none or more chimeric antigen receptor (CAR-T cell), wherein at least one chimeric antigen receptor specifically binds an incompatible BG-A antigen expressed on a cancer cell.

29. A T cell of claim 28, wherein the chimeric antigen receptor comprises one or more CDRs from the heavy chain variable region comprising SEQ ID NO:4 and/or one or more CDRs from the light chain variable region comprising SEQ ID NO:2.

30.-31. (canceled)

32. An antibody conjugate composition comprising an anti-incompatible BG-A antibody and one or more therapeutic agent or detectable label.

33. The antibody composition of claim 32, wherein the anti-incompatible BG-A antibody is CRC-A1.

34. The antibody composition of claim 32, wherein the anti-incompatible BG-A antibody comprises one or more CDRs from the heavy chain variable region comprising SEQ ID NO:4 and/or one or more CDRs from the light chain variable region comprising SEQ ID NO:2.

35. The antibody composition of claim 32, wherein the one or more therapeutic agent is a chemotherapeutic agents selected form methotrexate, adriamicin, vinca alkaloids (vincristine, vinblastine, etoposide), doxorubicin, melphalan, mitomycin C, chlorambucil, daunorubicin, other intercalating agents or combinations thereof.

36.-37. (canceled)

38. The CAR-T cell of claim 28, wherein the chimeric antigen receptor(s) specifically bind at least one antigen expressed on a skin cancer cell, a breast cancer cell, a lung cancer cell, a head and neck cancer cell, a prostate cancer cell, a esophageal cancer cell, a tracheal cancer cell, a brain cancer cell, a liver cancer cell, a bladder cancer cell, a stomach cancer cell, a pancreatic cancer cell, an ovarian cancer cell, an uterine cancer cell, a cervical cancer cell, a testicular cancer cell, a colon cancer cell, a rectal cancer cell, or a blood cancer cell.

39. The antibody of claim 32, wherein the antibody is selected from the group consisting of a single-chain antibody, an antibody fragment, a chimeric antibody, or a humanized antibody.

40. The antibody of claim 32, wherein the detectable label is selected from enzymes, radioactive isotopes, fluorescent compounds, chemical compounds, and bioluminescent proteins.

41. The method of claim 1, wherein soluble incompatible BG-A antigen and exosomal incompatible BG-A antigen are measured in the biological sample.
